(19) United States
(12) Patent Application Publication
(10) Pub. No.: US 2024/0385198 A1
(43) Pub. Date: Nov. 21, 2024

(54) STING MODULATOR COMPOSITIONS AND METHODS OF USE AND DETECTION THEREOF

(71) Applicant: University of South Florida, Tampa, FL (US)

(72) Inventor: Wayne Charles Guida, Saint Pete Beach, FL (US)

(21) Appl. No.: 18/594,993

(22) Filed: Mar. 4, 2024

Related U.S. Application Data
(60) Provisional application No. 63/488,037, filed on Mar. 2, 2023.

(51) Int. Cl. G01N 33/68

(52) U.S. Cl. CPC G01N 33/6845; G01N 2333/70503; G01N 2500/04

(57) ABSTRACT

The present disclosure is directed to compounds of Formula (I), (II), (III), and/or (IV), or pharmaceutically acceptable salts, that modulate stimulator of interferon genes (STING), compositions comprising such compounds, and methods of using same for detection and for treatment of disorders such as cancer and autoimmune disease.

### CROSS-REFERENCE TO RELATED APPLICATIONS

This application claims priority to U.S. Provisional Application No. 63/488,037, filed on Mar. 2, 2023, which is incorporated by reference herein in its entirety.

### FEDERALLY SPONSORED RESEARCH OR DEVELOPMENT

This invention was made with government support under Grant No. NIH/NIAID 1R21AI149450-01, awarded by the National Institutes of Health. The government has certain rights in the invention.

### BACKGROUND

Cancer is one of the world's most dreaded diseases. Tumor cells are hard to eliminate due their aberrant genetics, which results in uncontrolled growth. For example, “cold tumors” are a type of tumor that is not recognized and eradicated by the immune system. The STING (STimulator of INterferon Genes) pathway is involved in the innate immune response, which can help combat cancer, as well as cause certain autoimmune disorders such as systemic lupus erythematosus (SLE) and other diseases that are associated with an accumulation of nucleic acids in the cytoplasm.

STING-mediated production of IFN-β within the tumor microenvironment can result in activation of tumor antigen-specific CD8⁺ T-cell immunity that can lead to tumor regression. Mechanistic studies have shown that STING induced anti-tumor immunity is likely due to a pro-inflammatory cytokine response as well as the tumor specific CD8⁺ T-cellular response. STING activation by STING agonists should result in innate T-cell mediated anti-tumor immunity in the tumor microenvironment and have significant potential as a therapeutic strategy for the treatment of patients with advanced solid tumors. On the other hand, inhibition of STING (by STING antagonists) would lead to a decreased production of IFN-β and other Interferon Stimulated Genes (ISG) which are comprised of approximately 300 cytokines induced by the transcription factors interferon regulatory factor 3 (IRF3) and nuclear factor kappa-light-chain-enhancer of activated B cells (NF-KB). Inhibiting STING could have implications in the treatment of autoimmune disease such as lupus erythematosus.

Recently, there has been interest in developing agonists to increase activity of the STING pathway as a modality for cancer treatment. See e.g., WO2021042024A1, which is incorporated by reference in its entirety. Most STING agonists developed to date have been cyclic di-nucleotides (CDNs). In contrast, rather than activating STING to provoke an immune anti-tumor response, STING antagonists reduce anti-DNA antibody production resulting from abnormal removal of cytoplasmic DNA in immune cells. This leads to the accumulation of autoantibodies, chronic inflammation, and organ dysfunction that are hallmarks of SLE. In addition to SLE, accumulation of abnormal levels of cytoplasmic or lysosomal DNA (leading to STING activation) relate to several other diseases, including viral infections.

There is a need for the development of potent STING antagonistic and agonistic compositions for detection and treatment of autoimmune disorders and cancer.

### SUMMARY

The present application is based, in part, on the discovery of compositions capable of interaction with STING by computational methods. Initial screening using thermal scanning fluorimetry (thermal shift), THP-1 cell luciferase assay for interferon regulatory factor 3 (IRF3), and surface plasmon resonance (SPR) indicated that the composition had multiple interactions with STING at different concentrations. In order to move the composition into drug optimization, the disclosure includes compositions and methods based on methods that aim towards (1) development of a time and cost-effective assay for rapid screening of multiple analyte compositions; (2) design of a protocol capable of detecting composition interaction with STING at high and low concentrations simultaneously; and (3) overcoming detector limitations to be able to detect interactions within the femtomolar and attomolar range.

Some embodiments provide a method for detecting the affinity between stimulator of interferon genes (STING) and a compound (also referred to herein as an “analyte” or an “analyte compound”), the method comprising:
(a) contacting STING with 2′,3′-cGAMP to generate a first mixture, wherein the mixture comprises a detectable label;
(b) contacting the first mixture with the compound to form a second mixture; and
(c) detecting the detectable label using a microscale thermophoresis (MST) instrument.

Some embodiments provide a method of treating cancer comprising administering a therapeutically effective amount of a composition of Formula (I), (II), (III), and/or (IV), or a pharmaceutically acceptable salt(s) thereof, to a subject in need thereof.

Some embodiments provide a method of treating an autoimmune disorder comprising administering a therapeutically effective amount of a composition of Formula (I), (II), (III), and/or (IV), or a pharmaceutically acceptable salt(s) thereof.

In one aspect, disclosed herein is a method of treating cancer in a subject in need thereof, the method comprising administering a therapeutically effective amount of a composition, or a pharmaceutically acceptable salt thereof, wherein the composition comprises:

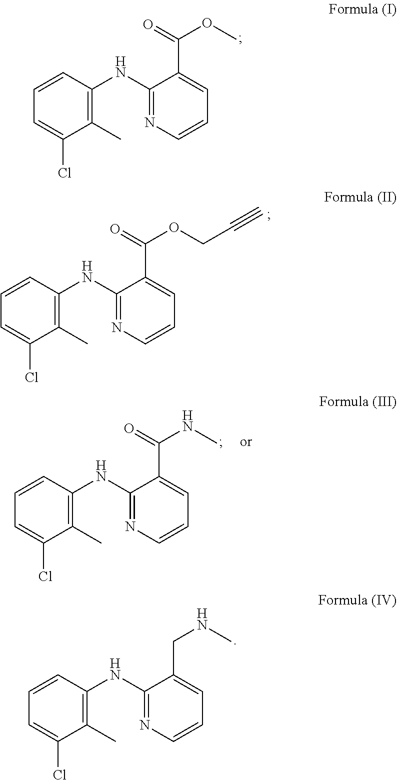 [SMILES: COC(=O)c1cccnc1Nc1cccc(Cl)c1C ; C#CCOC(=O)c1cccnc1Nc1cccc(Cl)c1C ; CNC(=O)c1cccnc1Nc1cccc(Cl)c1C ; CNCc1cccnc1Nc1cccc(Cl)c1C]

In some instances, the subject has been identified as having the cancer. In some instances, the cancer is a solid tumor. In some instances, the cancer is a blood cancer. In some instances, the cancer aberrantly expresses stimulator of interferon genes (STING). In some instances, the composition is administered to the subject at least two, three, four, five, six, seven, eight, nine, or ten times. In some instances, the cancer is melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplastic syndrome, multiple myeloma, transitional cell carcinoma, neuroblastoma, plasma cell neoplasms, Wilm's tumor, or hepatocellular carcinoma. In some instances, the subject is administered at least about 0.05 to about 1000 mg/kg of the composition. In some instances, the composition is administered orally, topically subcutaneously, intramuscularly, parenterally, intravenously or intradermally.

In another aspect, disclosed herein is a method of treating an autoimmune disorder in a subject in need thereof, the method comprising administering a therapeutically effective amount of a composition, or a pharmaceutically acceptable salt thereof, wherein composition comprises:

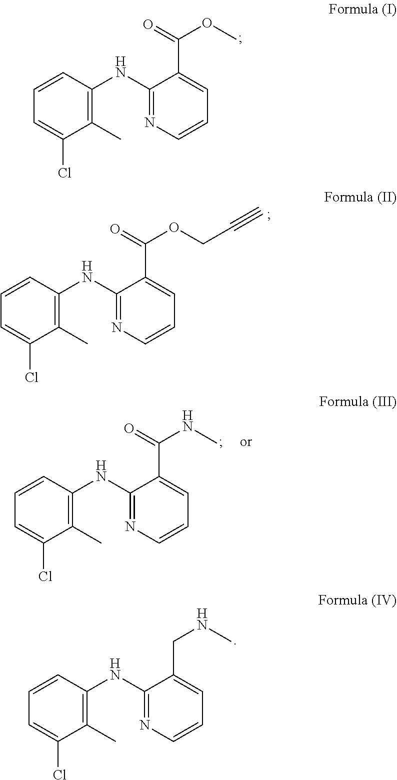 [SMILES: COC(=O)c1cccnc1Nc1cccc(Cl)c1C ; C#CCOC(=O)c1cccnc1Nc1cccc(Cl)c1C ; CNC(=O)c1cccnc1Nc1cccc(Cl)c1C ; CNCc1cccnc1Nc1cccc(Cl)c1C]

In some instances, the subject has been identified as having the autoimmune disorder. In some instances, the autoimmune disorder results in aberrantly expressed stimulator of interferon genes (STING). In some instances, the composition is administered to the subject at least two, three, four, five, six, seven, eight, nine, or ten times. In some instances, the autoimmune disease is selected from systemic lupus erythematosus (SLE), type 1 diabetes, rheumatoid arthritis, psoriatic arthritis, psoriasis, multiple sclerosis, inflammatory bowel disease, Addison's disease, Graves' disease, Sjogren's syndrome, thyroiditis, Myasthenia gravis, autoimmune vasculitis, pernicious anemia, or celiac disease. In some instances, the autoimmune disorder is systemic lupus erythematosus (SLE). In some instances, the subject is administered at least about 0.05 to about 1000 mg/kg of the composition. In some instances, the composition is administered orally, topically subcutaneously, intramuscularly, parenterally, intravenously or intradermally.

In yet another aspect, disclosed herein is a method for detecting the affinity between stimulator of interferon genes (STING) and 2′,3′-cGAMP, the method comprising: (a) mixing a biological sample comprising the STING with the 2′,3′-cGAMP, generating a mixture, wherein the mixture comprises a detectable label; (b) purifying the mixture; and (c) detecting the detectable marker obtained in the step using an microscale thermophoresis (MST) instrument. In some instances, the detectable label is a fluorescent label. In some instances, the biological sample is taken from a subject having cancer or from a subject having an autoimmune disorder.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A and FIG. 1B show 15 min incubation of 100 nM STING and drug candidate in 1:10 series dilution from concentration 100 mM to 100zM with (A) and without (B) 50 nM 2′,3′-cGAMP, respectively.

FIG. 2A, FIG. 2B, and FIG. 2C show dose-response curves for the compounds of Formula (I), (III), and (IV), respectively at 15 min, 30 min, and 45 min incubation time.

FIG. 3A and FIG. 3B shows MST analysis of 100 nM STING with 50 nM 2′,3′-cGAMP and the compound of Formula (I) in 1:10 series dilution from 100 mM to 100 zM. Several nonlinear regression curves can be seen, with three separate affinities (high, mid, and low) EC50 shown in FIG. 3B.

FIG. 4. shows MST analysis of 100 nM STING with 50 nM 2′,3′-cGAMP and the compound of Formula (II) in 1:10 series dilution from concentration 100 mM to 100 zM.

FIG. 5 depicts a plot showing the results of a microscale thermophoresis (MST) binding assay on compound 1.

FIG. 6 depicts a plot showing the results of a microscale thermophoresis (MST) binding assay on compound 4.

FIG. 7 depicts a plot showing the results of a microscale thermophoresis (MST) binding assay on compound 48.

FIG. 8 depicts a plot showing the results of a microscale thermophoresis (MST) binding assay on compound 49.

FIG. 9 depicts a plot showing the results of a microscale thermophoresis (MST) binding assay on compound 8.

FIG. 10 depicts a plot showing the results of a microscale thermophoresis (MST) binding assay on compound 9.

FIG. 11 depicts a plot showing the results of a microscale thermophoresis (MST) binding assay on compound 10.

FIG. 12 depicts a plot showing the results of a microscale thermophoresis (MST) binding assay on compound 11.

FIG. 13 depicts a plot showing the results of a microscale thermophoresis (MST) binding assay on compound 13.

FIG. 14 depicts a plot showing the results of a microscale thermophoresis (MST) binding assay on compound 14.

FIG. 15 depicts a plot showing the results of a microscale thermophoresis (MST) binding assay on compound 15.

FIG. 16 depicts a plot showing the results of a microscale thermophoresis (MST) binding assay on compound 16.

FIG. 17 depicts a plot showing the results of a microscale thermophoresis (MST) binding assay on compound 17.

FIG. 18 depicts a plot showing the results of a microscale thermophoresis (MST) binding assay on compound 19.

FIG. 19 depicts a plot showing the results of a microscale thermophoresis (MST) binding assay on compound 21.

FIG. 20 depicts a plot showing the results of a microscale thermophoresis (MST) binding assay on compound 23.

FIG. 21 depicts a plot showing the results of a microscale thermophoresis (MST) binding assay on compound 24.

FIG. 22 depicts a plot showing the results of a microscale thermophoresis (MST) binding assay on compound 26.

FIG. 23 depicts a plot showing the results of a microscale thermophoresis (MST) binding assay on compound 27.

FIG. 24 depicts a plot showing the results of a microscale thermophoresis (MST) binding assay on compound 29.

FIG. 25 depicts a plot showing the results of a microscale thermophoresis (MST) binding assay on compound 30.

FIG. 26 depicts a plot showing the results of a microscale thermophoresis (MST) binding assay on compound 32.

FIG. 27 depicts a plot showing the results of a microscale thermophoresis (MST) binding assay on compound 33.

FIG. 28 depicts a plot showing the results of a microscale thermophoresis (MST) binding assay on compound 35.

FIG. 29 depicts a plot showing the results of a microscale thermophoresis (MST) binding assay on compound 36.

FIG. 30 depicts a plot showing the results of a microscale thermophoresis (MST) binding assay on compound 38.

FIG. 31 depicts a plot showing the results of a microscale thermophoresis (MST) binding assay on compound 39.

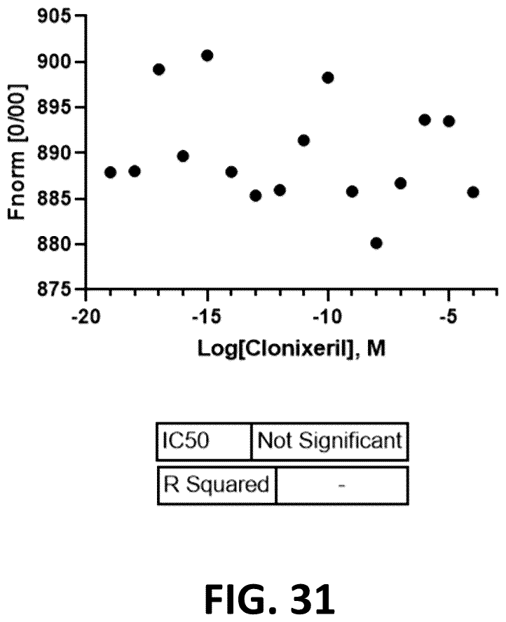

FIG. 32 depicts a plot showing the results of a microscale thermophoresis (MST) binding assay on compound 40.

FIG. 33 depicts a plot showing the results of a microscale thermophoresis (MST) binding assay on compound 41.

### DETAILED DESCRIPTION

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For purposes of the present disclosure, the following terms are defined.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” and “approximately” as used herein shall generally mean an acceptable degree of error for the quantity measured given the nature or precision of the measurements. Typical, exemplary degrees of error are within 10% or within 5% of a given value or range of values. Any reference to “about X” specifically indicates at least the values X, 0.95×, 0.96×, 0.97×, 0.98×, 0.99×, 1.01×, 1.02×, 1.03×, 1.04×, and 1.05×. Thus, “about X” is intended to provide written description support for a claim limitation of, e.g., “0.98×.” The terms “about” and “approximately,” particularly in reference to a given quantity, encompass and describe the given quantity itself.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 5, 10, or 15 mg” is equivalent to “about 5, about 10, or about 15 mg.”

As used herein, the terms “therapy,” “treating,” or “treatment” encompass the treatment of a disease state in a mammal, particularly in a human, and include: (a) preventing or delaying the occurrence of the disease state in a mammal, in particular, when such mammal is predisposed to the disease state but has not yet been diagnosed as having it; (b) inhibiting the disease state, i.e., arresting its development; and/or (c) achieving a full or partial reduction of the symptoms or disease state, and/or alleviating, ameliorating, lessening, or curing the disease or disorder and/or its symptoms.

“Administering” or “administration” refer to the physical introduction of a therapeutic agent to a subject, using any of the various methods and delivery systems known to those skilled in the art. Routes of administration can include oral, intravenous, intranasal, intramuscular, subcutaneous, intraperitoneal, spinal or other parenteral routes of administration, for example by injection or infusion (e.g., intravenous infusion). Administration can also be performed, for example, once, a plurality of times, and/or over one or more extended periods.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human.

An “effective amount” or “therapeutically effective amount” of a therapeutic agent is any amount of the drug that, when used alone or in combination with one or more additional therapies, slowing down the onset of a psychiatric disorder or promotes regression of the disorder evidenced by a decrease in severity of disorder symptoms, an increase in frequency and duration of disorder symptom-free periods, or a ameliorating an impairment or disability due to the disorder affliction. The ability of one or more additional therapies to promote disorder regression can be evaluated using a variety of methods known to the skilled practitioner, such as in human subjects during clinical trials, in animal model systems predictive of efficacy in humans, or by assaying the activity of the agent in in vitro assays. A therapeutically effective amount is intended to include an amount of a composition of the present disclosure that is effective when administered alone or in combination to inhibit STING-related conditions, diseases, or disorders.

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail herein.

### Methods of Detection

The present disclosure provides methods of detecting and analyzing interactions between molecules (e.g., STING genes and a signaling ligand such as 2′,3′-cGAMP and/or an analyte compound). In some instances, as exemplified in the Examples, the methods of detecting include microscale thermophoresis (MST). The rate at which fluorescently tagged molecules move away from an application of heat is affected by molecular size (differences suggesting that a binding event has occurred) or shape (differences suggesting that a conformation change has taken place). The present methods are designed to provide additional analytical data for drug discovery screening (e.g., a stability assessment for one hour) and whether multiple binding events occur over a wide range of analyte concentrations. The methods also enable the detection of low (e.g., femtomolar and attomolar) concentrations of analyte compounds not possible using conventional methods.

In some instances, the methods of detecting and analyzing interactions between molecules (e.g., STING genes and a signaling ligand such as 2′,3′-cGAMP and/or an analyte compound) include one or more steps of purifying the composition, incubating the molecules for a period of time (e.g., an hour), and then measuring the interaction between the molecules. In some instances, samples can be loaded into Monolith NT.115 capillaries and run on NanoTemper Pico instrument.

In some instances, the results of the methods of detecting and analyzing can include corroboration via a second (or more) assay. For instance, the results of the methods can be confirmed using Western blot, isothermal calorimetry (ITC), dynamic light scattering (DLS), surface plasmon resonance (SPR), and polymerase chain reactions (PCR) using conventional methods.

Some embodiments herein provide a method for detecting the affinity between stimulator of interferon genes (STING) and a compound (also referred to herein as an “analyte” or an “analyte compound”), the method comprising:
(a) contacting STING with 2′,3′-cGAMP to generate a first mixture, wherein the mixture comprises a detectable label;
(b) contacting the first mixture with the compound to form a second mixture; and
(c) detecting the detectable label using a microscale thermophoresis (MST) instrument.

In some embodiments, the first mixture is a buffered solution.

In some embodiments, the first mixture is a solution in 4-(2-hydroxyethyl)-1-piperazineethanesulfonic acid (HEPES) buffered saline surfactant P20 (HBS-P) buffer. In some embodiments, surfactant P20 is polysorbate 20.

In some embodiments, the concentration of the STING in the HBS-P buffer is about 1 nM to about 1 μM (e.g., about 1 nM to about 800 nM, about. In some embodiments, the concentration of the STING in the HBS-P buffer is about 200 nM.

In some embodiments, step (a) comprises contacting a dye with STING.

In some embodiments, the dye is a fluorescent dye.

In some embodiments, the fluorescent dye is NTAAtto 488 dye.

In some embodiments, the detectable label is a fluorescent label.

In some embodiments, contacting the first mixture with the compound comprises contacting the first mixture with a buffered solution of the compound.

In some embodiments, contacting the first mixture with the compound comprises contacting the first mixture with a solution of the compound in 4-(2-hydroxyethyl)-1-piperazineethanesulfonic acid (HEPES) buffered saline surfactant P20 (HBS-P) buffer.

In some embodiments, the solution comprises about 1% to about 5% (e.g., about 1%) dimethylsulfoxide (DMSO).

In some embodiments, the concentration of the buffered solution of the compound is less than 200 μM.

In some embodiments, the concentration of the buffered solution of the compound is less than 1 μM.

In some embodiments, the concentration of the buffered solution of the compound is less than 1 nM.

In some embodiments, the concentration of the buffered solution of the compound is less than 1 pM.

In some embodiments, the concentration of the buffered solution of the compound is less than 1 fM.

In some embodiments, the concentration of the buffered solution of the compound is less than 1 aM.

In some embodiments, the concentration of the buffered solution of the compound is less than 1 zM.

In some embodiments, detecting the detectable label comprises measuring the normalized fluorescence as a function of the concentration of the solution of the compound.

In some embodiments, the compound is a compound of Formula (I), (II), (III), or (IV).

In some embodiments, the compound is a compound selected from the compounds disclosed in Table A.

### Compositions

The present disclosure also provides compositions and methods of use thereof.

Some embodiments provide compositions (e.g., pharmaceutical compositions) comprising one of Formula (I), Formula (II), Formula (III), or Formula (IV), or a pharmaceutically acceptable salt thereof, and a pharmaceutically acceptable carrier; wherein the composition is formulated for intranasal administration.

Some embodiments, provide compositions of Formula (I), or pharmaceutically acceptable salts thereof. Formula (I) is shown below. In some instances, Formula (I) is referred to as Compound 3.

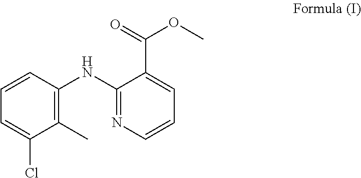 [SMILES: COC(=O)c1cccnc1Nc1cccc(Cl)c1C]

Some embodiments, provide compositions of Formula (II), or pharmaceutically acceptable salts thereof. Formula (II) is shown below. In some instances, Formula (I) is referred to as Compound 6.

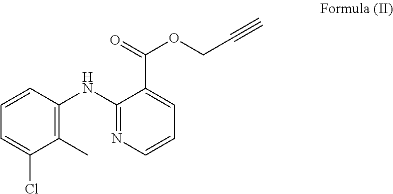 [SMILES: C#CCOC(=O)c1cccnc1Nc1cccc(Cl)c1C]

Some embodiments, provide compositions of Formula (III), or pharmaceutically acceptable salts thereof. Formula (III) is shown below. In some instances, Formula (I) is referred to as Compound 7.

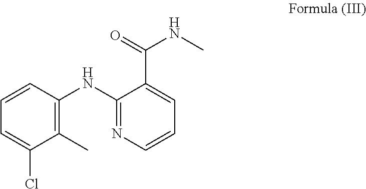 [SMILES: CNC(=O)c1cccnc1Nc1cccc(Cl)c1C]

Some embodiments, provide compositions of Formula (IV), or pharmaceutically acceptable salts thereof. Formula (IV) is shown below. In some instances, Formula (I) is referred to as Compound 31.

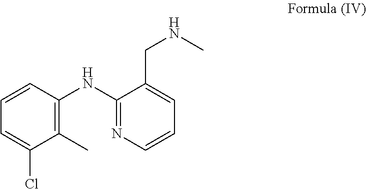 [SMILES: CNCc1cccnc1Nc1cccc(Cl)c1C]

The compositions may contain other therapeutic agents as described herein and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

In some embodiments, the composition further comprises a preservative. Exemplary preservatives include, but are not limited to parabens (e.g., alkyl parabens), benzyl alcohol, chlorobutanol, benzoic acid, sorbic acid, propylene glycol, quaternary ammonium salts (e.g., benzalkonium chloride and benzethonium chloride). In some embodiments, the preservative is benzalkonium chloride.

In some embodiments, the composition further comprises one or more excipients selected from the group consisting of surfactants, antioxidants, buffers, and absorption enhancing agents. Exemplary surfactants include, but are not limited to ionic, nonionic, and amphoteric surface active agents. For example, Tweens, PEGs, sorbitan esters, and ethoxylated fatty acids. In some embodiments, the composition further comprises a surfactant in an amount of about 1% to about 10% surfactant (w/v).

Exemplary antioxidants include, but are not limited to tocopherols, butyl hydroxytoluene, sodium metabisulfite, potassium metabisulfite, and ascorbyl palmitate. In some embodiments, the composition further comprises an antioxidant in an amount of about 0.001% to about 5% (w/w).

Exemplary absorption enhancing agents include, but are not limited to chitosan, caproic acid salts, and cyclopentadecalactone. In some embodiments, the composition further comprises an absorption enhancing agent in an amount of about 1% to about 10% (w/w).

Exemplary buffers include, but are not limited to citrate, phosphate, acetate, lactate, fumarate, tartrate, malate, and amino acid-based buffers. In some embodiments, the composition further comprises a buffer in an amount of about 0.1% to about 5% (w/w).

In some embodiments, the pharmaceutically acceptable carrier is water or saline.

Exemplary compositions for topical administration include a topical carrier such as PLASTIBASE® (mineral oil gelled with polyethylene).

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The composition may also be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

### Methods and Uses

The disclosure provides methods and uses of Formulae (I), (II), (III), or (IV), or a pharmaceutically acceptable salt thereof. When the terms “STING-associated condition” or “STING-associated disease or disorder” are used herein, each is intended to encompass all of the conditions identified above as if repeated at length, as well as any other condition that is affected by STING gene activity. The present disclosure provides at least one composition having Formula (I), (II), (III) or (IV), or a pharmaceutically acceptable salt thereof, for use in therapy for the treatment for treating such conditions.

Some embodiments provide a method for a treating cancer in a subject in need thereof, comprising administering a therapeutically effective amount of one of Formulae (I), (II), (III), or (IV), or a pharmaceutically acceptable salt thereof, to the subject.

In some instances, the cancer is a solid tumor cancer. In some instances, the cancer is a blood cancer. In some instances, the cancer aberrantly expresses stimulator of interferon genes (STING).

In some embodiments described herein, the cancer resolves faster relative to the resolution observed after treatment without one of Formulae (I), (II), (III), or (IV), or a pharmaceutically acceptable salt thereof. For example, in some embodiments, the size of a tumor may shrink relative to the size prior to treatment or relative to a control subject in which no treatment of Formulae (I), (II), (III), or (IV), or a pharmaceutically acceptable salt thereof, was provided. In some embodiments, the tumor size shrinks to about 90%, about 80%, about 70%, about 60%, about 50%, about 40%, about 30%, about 20%, about 10%, about 5%, or about 0% of the original size prior to treatment.

Also disclosed herein are methods that include treatment with a compositions comprising one of Formula (I), (II), (III), and/or (IV) in combination with different forms of treatment to treat each subject with cancer. Combination methods include, for example, surgery, radiotherapy, and chemotherapeutic agents, such as other kinase inhibitors, signal transduction inhibitors and/or monoclonal antibodies. For example, a surgery may be open surgery or minimally invasive surgery. In some instances, the combination methods includes surgery, radiotherapy, chemotherapy, toxin therapy, immunotherapy, cryotherapy or gene therapy.

Compositions of Formula (I), (II), (III), and/or (IV), or a pharmaceutically acceptable salt thereof therefore may also be useful as adjuvants to cancer treatment, that is, they can be used in combination with one or more additional therapies or therapeutic agents, for example, a chemotherapeutic agent that works by the same or by a different mechanism of action. In some embodiments, a composition of Formula (I), (II), (III), and/or (IV), or a pharmaceutically acceptable salt thereof, can be used prior to administration of an additional therapeutic agent or additional therapy. For example, a subject in need thereof can be administered one or more doses of a composition of Formula (I), (II), (III), and/or (IV), or a pharmaceutically acceptable salt thereof for a period of time and then undergo at least partial resection of the tumor. In some embodiments, the treatment with one or more doses of a compound of Formula (I), (II), (III), and/or (IV), or a pharmaceutically acceptable salt thereof reduces the size of the tumor (e.g., the tumor burden) prior to the at least partial resection of the tumor. In some embodiments, a subject in need thereof can be administered one or more doses of a composition of Formula (I), (II), (III), and/or (IV), or a pharmaceutically acceptable salt thereof for a period of time and under one or more rounds of radiation therapy. In some embodiments, the treatment with one or more doses of a composition of Formula (I), (II), (III), and/or (IV), or a pharmaceutically acceptable salt thereof reduces the size of the tumor (e.g., the tumor burden) prior to the one or more rounds of radiation therapy.

In some embodiments of any of the methods described herein, the composition of Formula (I), (II), (III), and/or (IV), or a pharmaceutically acceptable salt thereof, is administered in combination with an effective amount of at least one additional therapeutic agent selected from one or more additional therapies or therapeutic (e.g., chemotherapeutic) agents.

Non-limiting examples of receptor tyrosine kinase (e.g., Trk) targeted therapeutic agents, include afatinib, cabozantinib, cetuximab, crizotinib, dabrafenib, entrectinib, erlotinib, gefitinib, imatinib, lapatinib, lestaurtinib, nilotinib, pazopanib, panitumumab, pertuzumab, sunitinib, trastuzumab, 1-((3 S,4R)-4-(3-fluorophenyl)-1-(2-methoxyethyl) pyrrolidin-3-yl)-3-(4-methyl-3-(2-methylpyrimidin-5-yl)-1-phenyl-1H-pyrazol-5-yl) urea, AG 879, AR-772, AR-786, AR-256, AR-618, AZ-23, AZ623, DS-6051, Go 6976, GNF-5837, GTx-186, GW 441756, LOXO-101, MGCD516, PLX7486, RXDX101, VM-902A, TPX-0005, TSR-011, GNF-4256, N-[3-[2,3-dihydro-2-oxo-3-(1H-pyrrol-2-ylmethylene)-1H-indol-6-yl]amino]-4-methylphenyl]-N′-[2-fluoro-5-(trifluoromethyl) phenyl]-urea, AZ623, AZ64, (S)-5-Chloro-N2-(1-(5-fluoropyridin-2-yl) ethyl)-N4-(5-isopropoxy-1H-pyrazol-3-yl) pyrimidine-2, 4-diamine, AZD7451, CEP-751, CT327, sunitinib, GNF-8625, and (R)-1-(6-(6-(2-(3-fluoro-phenyl) pyrrolidin-1-yl) imidazo[1,2-b]pyridazin-3-yl)-[2,4′-bipyridin]-2′-yl) piperidin-4-ol.

Provided herein are methods of treating a subject having a cancer (e.g., any of the cancers described herein) and previously administered a multi-kinase inhibitor (MKI) or a target-specific kinase inhibitor (e.g., a BRAF inhibitor, an EGFR inhibitor, a MEK inhibitor, an ALK inhibitor, a ROS1 inhibitor, a MET inhibitor, an aromatase inhibitor, a RAF inhibitor, a RET inhibitor, or a RAS inhibitor) (e.g., as a monotherapy) that include: administering to the subject (i) an effective dose of a composition of Formula (I), (II), (III), and/or (IV), or a pharmaceutically acceptable salt thereof as a monotherapy, or (ii) an effective dose of a composition of Formula (I), (II), (III), and/or (IV), or a pharmaceutically acceptable salt thereof, and an effective dose of the previously administered MKI or the previously administered target-specific kinase inhibitor.

Also provided herein is a method of inhibiting mammalian cell proliferation, in vitro or in vivo, the method comprising contacting a mammalian cell with an effective amount of a composition of Formula (I), (II), (III), and/or (IV), or a pharmaceutically acceptable salt thereof, or a pharmaceutical composition thereof as defined herein.

In some embodiments, the disorder or disease is cancer. In some instances, the cancer is a solid tumor cancer. In some embodiments, the cancer is an adenocarcinoma, a bile duct (biliary) cancer, a bladder cancer, a bone cancer, a breast cancer (e.g., a triple-negative breast cancer, a Her2-negative breast cancer), a carcinoid cancer, a cervical cancer, a cholangiocarcinoma, a colorectal cancer, a colon cancer, an endometrial cancer, a glioma, a head and neck cancer, a head and neck squamous cell cancer, a leukemia, a liver cancer, a lung cancer, a non-small cell lung cancer, a small cell lung cancer, a lymphoma, a melanoma, an oropharyngeal cancer, an ovarian cancer, a pancreatic cancer, a prostate cancer, a metastatic castration-resistant prostate carcinoma, a renal cancer, a sarcoma, a skin cancer, a squamous cell cancer, a stomach cancer, a testis cancer, a thyroid cancer, a urogenital cancer, or a urothelial cancer. In some instances, the lung cancer is non-small cell lung cancer. In some instances, the lymphoma is diffuse large B cell lymphoma or non-Hodgkin's lymphoma. In some instances, the esophageal cancer is esophageal adenocarcinoma, esophageal squamous cell carcinoma, or gastroesophageal junction carcinoma. In some embodiments, the cancer is a chemotherapy or radio-resistant cancer.

Some embodiments provide a method for a treating autoimmune disorder in a subject in need thereof, comprising administering a therapeutically effective amount of one of Formulae (I), (II), (III), or (IV), or a pharmaceutically acceptable salt thereof, to the subject.

In some embodiments described herein, the autoimmune disorder resolves faster after treatment of one of Formulae (I), (II), (III), or (IV), or a pharmaceutically acceptable salt thereof, relative to no treatment of one of Formulae (I), (II), (III), or (IV), or a pharmaceutically acceptable salt thereof. In some embodiments, the autoimmune disorder resolves from about 1.2× to about 10× faster after treatment of one of Formulae (I), (II), (III), or (IV), or a pharmaceutically acceptable salt thereof,

Autoimmune disease includes diseases or disorders arising from and directed against an individual's own tissues or organs or manifestation thereof or a condition resulting there from. In one embodiment, it refers to a condition that results from, or is aggravated by, the production by T cells that are reactive with normal body tissues and antigens.

More particularly, the specific conditions or diseases that may be treated with the compositions include, without limitation, asthma, allergies, glomerulonephritis, rheumatoid arthritis, systemic lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, myasthenia gravis, multiple sclerosis, inflammatory bowel disease, ulcerative colitis, Crohn's disease, psoriasis, graft vs. host disease, pancreatic β-cell disease; rheumatoid spondylitis, allograft rejections, ulcerative colitis, dry eye and pemphigus vulgaris. Preferred methods of treatment are those wherein the condition is selected from allograft rejection, rheumatoid arthritis, psoriasis, ankylosing spondylitis, psoriatic arthritis, multiple sclerosis, lupus and dry eye.

Examples of autoimmune diseases or disorders include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano-or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes). Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and giant cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenia purpura (ITP) including chronic or acute ITP, scleritis such as idiopathic ceratoscleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FS GS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

In some embodiments, the autoimmune disorder is SLE. In some embodiments, treating SLE comprises treating joint pain, heart disease, kidney disease, and photosensitivity in the subject. In some embodiments, treating the autoimmune disorder comprises antagonizing STING, as described herein. In some embodiments, treating the autoimmune disorder comprises reducing expression of IFN-b in the subject, as described herein. In some embodiments, treating the autoimmune disorder comprises reducing expression of one or more cytokines (e.g., two or more, five or more, ten or more, for example up to twenty or fifty cytokines. In some embodiments, the one or more cytokines are induced by IRF3. In some embodiments, the one or more cytokines are induced by NF-KB. In some embodiments, the one or more cytokines are induced by IRF3 and NF-KB. In some embodiments, treating the subject comprises reducing expression of STAT1, STAT2, Cig5, GIP3, IRF7, IFIT4, Ly6E, MX1, OAS3 and IFI27 in the subject. In some embodiments, treating the subject comprises reducing expression of TOR1B, Clorf29, FAM3B, OAS3, USP18, OAS1, Siglec-1, GBP5, IFIT5, IFIT2, IFRG28, IFIT1, PRKR (EIF2AK1), IL1RN, OASL, OAS1, LGALS3BP, OASL, IFIH1 (MDA5), ZBP1, C1QB, CEB1, GBP1, BST2, IFI44, IFI27, GBP2, EPSTI1, CARD15, IFI35, SOCS1, TAP1, XAF1, SP110, OAS2, STAT1, ABCA1, IFIT4, PLSCR1, Cig5, ISG95, STAT2, RIG-I (DDX58), MX2, LGP2, IRF7, ADD45B, SCOTIN, PARP9 (BAL), MT2A, NT5C3 (PN-1), MX1, STAT1, ADAR, TRIM22, G1P2, SERPING1, STAT1, NUB1 (NYREN18), ISG20, LY6E, GIP3, and/or IFITM1 in the subject. In some embodiments, treating the subject comprises reducing expression of STAT1, STAT2, Cig5, G1P3, IRF7, IFIT4, Ly6E, MX1, OAS3 and/or IFI27 in the subject.

It is normal practice to use a combination of different forms of treatment to treat a subject with an autoimmune disorder. The other component(s) of such conjoint treatment or therapy in addition to compositions provided herein (e.g., the composition of Formula (I), (II), (III), and/or (IV), or pharmaceutically acceptable salts thereof) may be, for example, anti-inflammatory drugs, cytotoxic chemotherapeutic drugs, immunosuppressants, kidney support, anti-rheumatic drugs, monoclonal antibodies, and avoiding sun exposure.

In some embodiments of any of the methods described herein, the composition of Formula (I), (II), (III), and/or (IV), or a pharmaceutically acceptable salt thereof, is administered in combination with an effective amount of at least one anti-inflammatory drug selected from steroids (e.g., corticosteroids, hydrocortisone, prednisone, triamcinolone, betamethasone, dexamethasone, Prednisolone, methylprednisolone), ibuprofen, naproxen, aspirin, diclofenac, meloxicam, and tolmetin. The at least one anti-inflammatory drug may be a topical drug or treatment. Topical drugs or treatments include, but are not limited to, triamcinolone and fluocinolone.

In some embodiments of any of the methods described herein, the composition of Formula (I), (II), (III), and/or (IV), or a pharmaceutically acceptable salt thereof, is administered in combination with an effective amount of at least one cytotoxic chemotherapeutic drug selected from arsenic trioxide, bleomycin, cabazitaxel, capecitabine, carboplatin, cisplatin, cyclophosphamide, cytarabine, dacarbazine, daunorubicin, docetaxel, doxorubicin, etoposide, fluorouracil, gemcitabine, irinotecan, lomustine, methotrexate, mitomycin C, oxaliplatin, paclitaxel, pemetrexed, temozolomide, and vincristine.

In some embodiments of any of the methods described herein, the composition of Formula (I), (II), (III), and/or (IV), or a pharmaceutically acceptable salt thereof, is administered in combination with an effective amount of at least one immunosuppressant selected from azathioprine, methotrexate, mycophenolate, imuran, azathioprine, Mycophenolate mofetil, Tacrolimus, Sirolimus, Everolimus, and Interferons.

In some embodiments of any of the methods described herein, the composition of Formula (I), (II), (III), and/or (IV), or a pharmaceutically acceptable salt thereof, is administered in combination with an effective amount of at least one anti-rheumatic drug selected from hydroxychloroquine, celecoxib, abatacept, adalimumab, anakinra, apremilast, baricitinib, certolizumab pegol, ciclosporin (Cyclosporin A), D-penicillamine, etanercept, filgotinib, golimumab, infliximab, leflunomide, methotrexate, minocycline, rituximab, sarilumab, secukinumab, sulfasalazine, tocilizumab, tofacitinib, and ustekinumab.

In some embodiments of any of the methods described herein, the composition of Formula (I), (II), (III), and/or (IV), or a pharmaceutically acceptable salt thereof, is administered in combination with an effective amount of at least one monoclonal antibody selected from abciximab, alefacept, alemtuzumab, basiliximab, bezlotoxumab, canakinumab, certolizumab pegol, cetuximab, daclizumab, denosumab, efalizumab, golimumab, inflectra, ipilimumab, ixekizumab, natalizumab, nivolumab, olaratumab, omalizumab, palivizumab, panitumumab, pembrolizumab, tocilizumab, trastuzumab, secukinumab, ustekinumab, belimumab, rituximab, infliximab, and adalimumab.

The compositions of this disclosure can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compositions of the present disclosure will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to treat the cancer.

The active composition may be effective over a wide dosage range and is generally ad ministered in a pharmaceutically effective amount. Optimal dosages to be administered can be readily determined by those skilled in the art. It will be understood, therefore, that the amount of the composition actually administered will usually be determined by a physician, and will vary according to the relevant circumstances, including the mode of administration, the actual composition administered, the strength of the preparation, the condition to be treated, and the advancement of the disease condition. In addition, factors associated with the particular subject being treated, including subject response, age, weight, diet, time of administration and severity of the subject's symptoms, will result in the need to adjust dosages.

The therapeutically-effective amount of a composition of the present disclosure may be determined by one of ordinary skill in the art, and includes exemplary dosage amounts for a mammal of from about 0.05 to 1000 mg/kg; 1-1000 mg/kg; 1-50 mg/kg; 5-250 mg/kg; 250-1000 mg/kg of body weight of active composition per day, which may be administered in a single dose or in the form of individual divided doses, such as from 1 to 4 times per day. It will be understood that the specific dose level and frequency of dosage for any particular subject may be varied and will depend upon a variety of factors, including the activity of the specific composition employed, the metabolic stability and length of action of that composition, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like.

In some embodiments, the compositions provided herein can be administered in an amount ranging from about 1 mg/kg to about 100 mg/kg. In some embodiments, the composition provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

In some embodiments, the compositions provided herein can be administered in an amount of about 10 mg twice a day (BID), 20 mg BID, about 40 mg BID, about 60 mg BID, about 80 mg BID, about 120 mg BID, about 160 mg BID, and about 240 mg BID. In some embodiments, each dose is administered at least six hours after the previous dose. In some embodiments, each dose is administered at least twelve hours after the previous dose.

Compositions of this disclosure can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compositions are typically administered in admixture with suitable pharmaceutical diluents, excipients, or carriers (collectively referred to herein as pharmaceutical carriers) suitably selected with respect to the intended form of administration, that is, oral tablets, capsules, elixirs, syrups and the like, and consistent with conventional pharmaceutical practices. Suitable pharmaceutical carriers are described in Remington's Pharmaceutical Sciences, Mack Publishing Company, a standard reference text in this field.

In another embodiment, the disclosure also provides the use of a composition of Formulae (I), (II), (III), or (IV) for the manufacture of a medicament for the treatment of a cancer and/or an autoimmune disease.

Also disclosed herein are methods of producing any one of Formulae (I), (II), (III), or (IV). Methods known in the art can be used to produce any one of the compositions. The process can include the selection of readily available starting materials, thereby ensuring a cost-effective and sustainable source and allow for high selectivity and yield. Additionally, the method optimizes reaction kinetics and enhance the efficiency of the synthesis process. Notably, this approach significantly reduces the generation of unwanted by-products and minimizes the environmental impact associated with traditional synthesis methods.

One skilled in the art will recognize that both in vivo and in vitro trials using suitable, known and generally accepted cell and/or animal models are predictive of the ability of a test composition to treat or prevent a given disorder. One skilled in the art will further recognize that human clinical trials including first-in human, dose ranging and efficacy trials, in healthy subjects and/or those suffering from a given disorder, can be completed according to methods well known in the clinical and medical arts.

Provided herein are pharmaceutical kits useful, for example, in the treatment of STING-associated diseases or disorders, such as cancer, which include one or more containers containing a pharmaceutical composition comprising an effective amount of a composition provided herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### Exemplary Embodiments

Embodiment 1. A method of treating cancer in a subject in need thereof, the method comprising administering a therapeutically effective amount of a composition, or a pharmaceutically acceptable salt thereof, wherein the composition comprises:

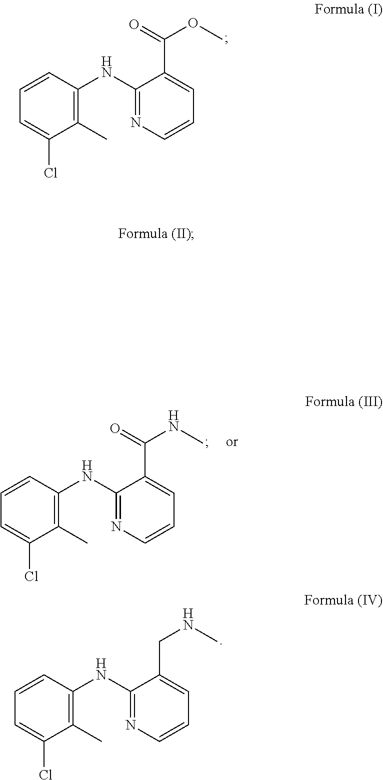 [SMILES: COC(=O)c1cccnc1Nc1cccc(Cl)c1C ; CNC(=O)c1cccnc1Nc1cccc(Cl)c1C ; CNCc1cccnc1Nc1cccc(Cl)c1C]

Embodiment 2. The method of embodiment 1, wherein the subject has been identified as having the cancer.

Embodiment 3. The method of embodiment 1, wherein the cancer is a solid tumor.

Embodiment 4. The method of embodiment 1, wherein the cancer is a blood cancer.

Embodiment 5. The method of embodiment 1, wherein the cancer aberrantly expresses stimulator of interferon genes (STING).

Embodiment 6. The method of embodiment 1, wherein the composition is administered to the subject at least two, three, four, five, six, seven, eight, nine, or ten times.

Embodiment 7. The method of embodiment 1, wherein the cancer is melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplastic syndrome, multiple myeloma, transitional cell carcinoma, neuroblastoma, plasma cell neoplasms, Wilm's tumor, or hepatocellular carcinoma.

Embodiment 8. The method of embodiment 1, wherein the subject is administered at least about 0.05 to about 1000 mg/kg of the composition.

Embodiment 9. The method of embodiment 1, wherein the composition is administered orally, topically subcutaneously, intramuscularly, parenterally, intravenously or intradermally.

Embodiment 10. A method of treating an autoimmune disorder in a subject in need thereof, the method comprising administering a therapeutically effective amount of a composition, or a pharmaceutically acceptable salt thereof, wherein composition comprises:

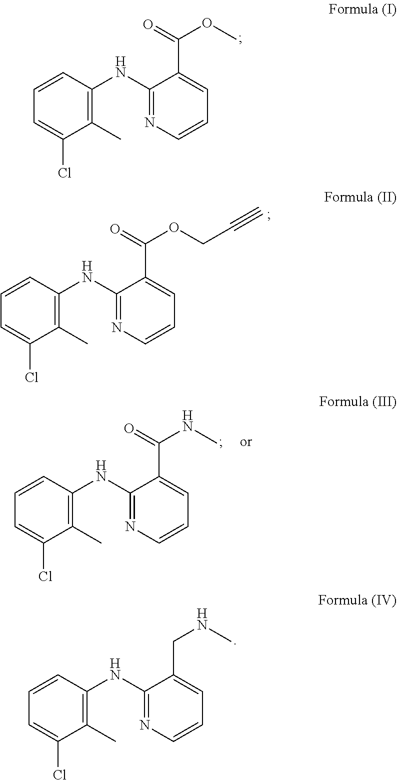 [SMILES: COC(=O)c1cccnc1Nc1cccc(Cl)c1C ; C#CCOC(=O)c1cccnc1Nc1cccc(Cl)c1C ; CNC(=O)c1cccnc1Nc1cccc(Cl)c1C ; CNCc1cccnc1Nc1cccc(Cl)c1C]

Embodiment 11. The method of embodiment 10, wherein the subject has been identified as having the autoimmune disorder.

Embodiment 12. The method of embodiment 10, wherein the autoimmune disorder results in aberrantly expressed stimulator of interferon genes (STING).

Embodiment 13. The method of embodiment 10, wherein the composition is administered to the subject at least two, three, four, five, six, seven, eight, nine, or ten times.

Embodiment 14. The method of embodiment 10, wherein the autoimmune disease is selected from systemic lupus erythematosus (SLE), type 1 diabetes, rheumatoid arthritis, psoriatic arthritis, psoriasis, multiple sclerosis, inflammatory bowel disease, Addison's disease, Graves' disease, Sjogren's syndrome, thyroiditis, Myasthenia gravis, autoimmune vasculitis, pernicious anemia, or celiac disease.

Embodiment 15. The method of embodiment 14, wherein the autoimmune disorder is systemic lupus erythematosus (SLE).

Embodiment 16. The method of embodiment 10, wherein the subject is administered at least about 0.05 to about 1000 mg/kg of the composition.

Embodiment 17. The method of embodiment 10, wherein the composition is administered orally, topically subcutaneously, intramuscularly, parenterally, intravenously or intradermally.

Embodiment 18. A method for detecting the affinity between stimulator of interferon genes (STING) and 2′,3′-cGAMP, the method comprising:
(a) mixing a biological sample comprising the STING with the 2′,3′-cGAMP to generate a mixture, wherein the mixture comprises a detectable label;
(b) purifying the mixture; and
(c) detecting the detectable label using a microscale thermophoresis (MST) instrument.

Embodiment 19. The method of embodiment 18, wherein the detectable label is a fluorescent label.

Embodiment 20. The method of embodiment 18, wherein the biological sample is taken from a subject having cancer or from a subject having an autoimmune disorder.

### EXAMPLES

### Example 1. MST Measurement of STING Oligomerization by 2′,3′-cGAMP as a Means of Determining Sub-Picomolar Interactions of Compounds with STING

Temperature Related Intensity Change (TRIC) is a biophysical technique that measures the strength of the interaction between two molecules by detecting a variation in the fluorescence signal of a fluorescently labeled or intrinsically fluorescent target as a result of an IR-laser induced temperature change. The extent of the temperature dependence is strongly related to the chemical environment of the fluorophore, which can be changed by the binding of a ligand to the target. Binding events below the picomolar level are difficult to detect because below this level, the ratio between target protein and analyte is too large for the detector to accurately measure movement. Herein describes a competition assay involving STING along with 2′,3′-cGAMP to detect analyte interaction with STING at sub-picomolar levels by measuring changes to oligomerization of STING.

The NanoTemper Pico instrument produces data in three separate phases: fluorescent count; normalized MST time-trace; and the change in normalized fluorescence (ΔFₙₒᵣₘ) result. The fluorescent count is done to ensure that there is no difference between overall fluorescence between fractions, that there is enough fluorescent signal present for the detector to accurately give a reading, but not too much as to negatively affect migration during thermophoresis. It also gives indication if there is aggregation or adsorption within samples. The normalized MST time-trace gives an indication of the movement of molecules by thermophoresis within the detector sight picture over a period of time (T−Jump). The final graph is a ΔFₙₒᵣₘ readout which is mathematically determined using the formula:

ΔFₙₒᵣₘ=Fₕₒₜ/F_{cold}
whereas Fₕₒₜ equals an area designated by the analyst, located within the T−Jump area and F_{cold} equals the area within the initial fluorescence area just before the infrared (IR) laser heats the capillary tube.

### General Procedure

STING R232 variant (human recombinant) protein was solubilized at 200 nM in HBS-P (0.01M HEPES pH 7.4, 0.15 NaCl, 0.005% v/v surfactant P20) along with 100 nM 2′,3′-cGAMP. 100 nM of NTAAtto 488 dye (blue; nitrilotriacetic acid complexed to Ni²⁺) was added and incubated for 1 hour at room temperature covered from light. The resulting mixture was centrifuged at 12,000×RPM for 10 minutes prior to use. A 1:10 series dilution from 200 mM to 200 zM was created using HBS-P with 1% DMSO. The dyed STING/2′,3′-cGAMP mixture was added to each sample in a 1:1 ratio, resulting in a static 100 nM STING, 50 nM 2′,3′-cGAMP, and a concentration range from 100 mM to 100zM. Samples were incubated for 15-minutes prior to load into Monolith NT.115 capillaries and ran on dianthus NT.23 NanoTemper Pico instrument (NanoTemper Technologies GmbH, München, Germany). Samples were run again at 30- and 45-minutes.

### Results

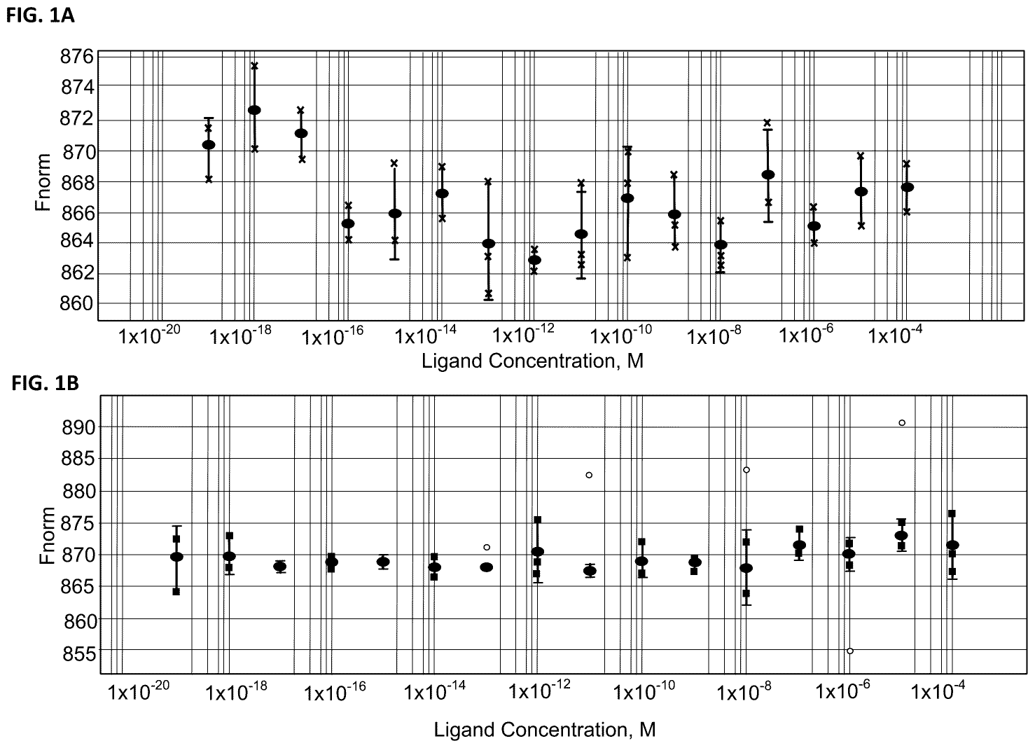

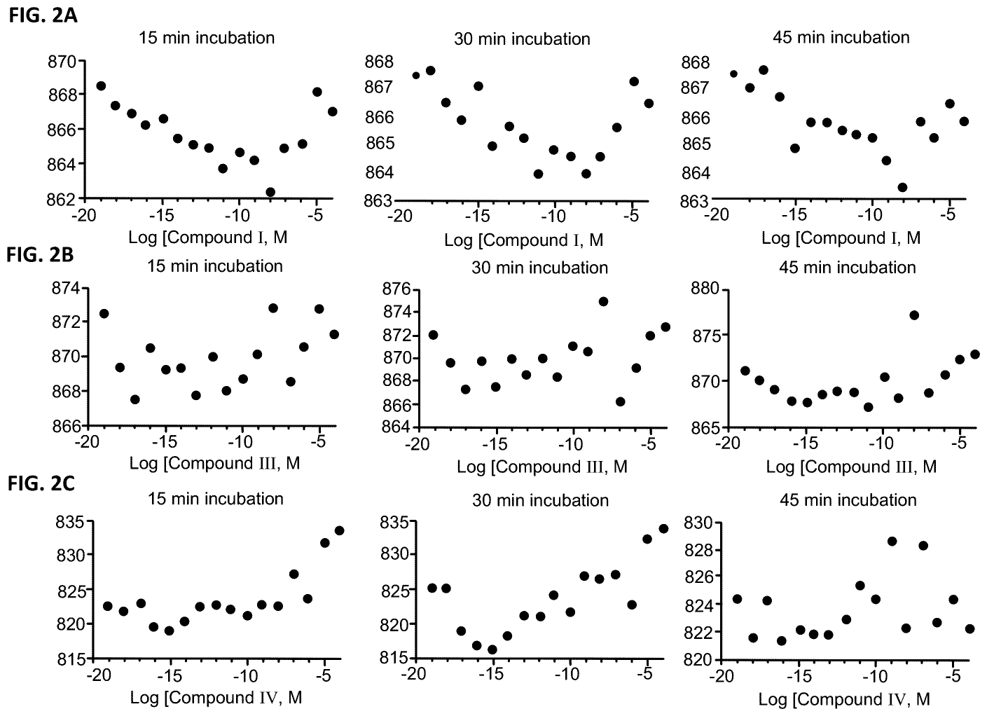

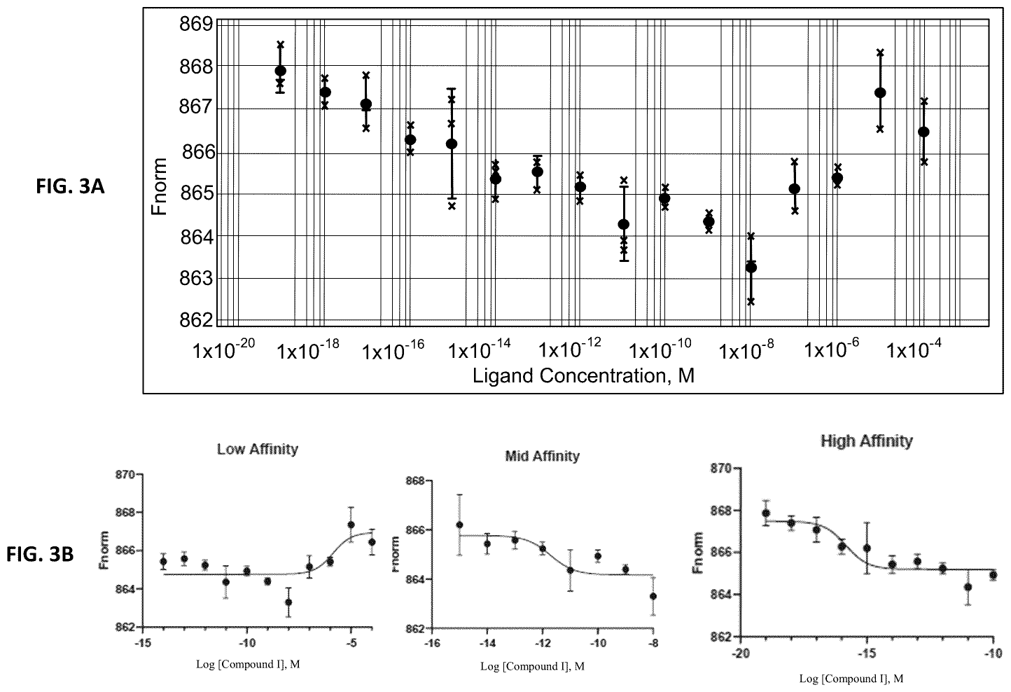

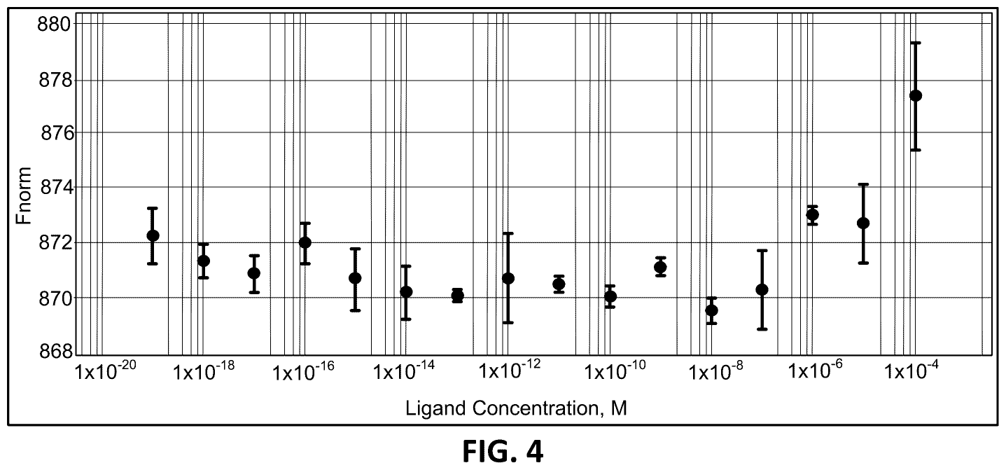

As shown in FIG. 1A, incubation of 100 nM STING with 50 nM 2′,3′-cGAMP and drug candidate for 15 minutes in 1:10 series dilution from concentration 100 mM to 100 zM resulted in a detectable change in the normalized fluorescence signal, while the same experiment without 2′,3′-cGAMP (FIG. 1B) resulted in no signal change in the lower concentration range. Incubation time has an effect on dose response for compounds interacting with STING. As shown in FIGS. 2A, 2B, and 2C, the compound of Formula (III) demonstrated a relatively slow interaction, while the compound of Formula (IV) showed a relatively rapid interaction with STING that was not maintained. These structure-kinetic relationships assist in optimization of drug candidates. A wide concentration range enables calculation of multiple EC₅₀ interactions with a single assay. For example, the compound of Formula (I) showed several nonlinear regression curves (FIG. 3A) from which affinities (high, mid, and low) EC₅₀ were calculated (FIG. 3B). The compound of Formula (II), on the other hand, showed only a low affinity EC50 (FIG. 4). Initial screening of compound using thermal scanning fluorimetry (thermal shift), THP-1 cell luciferase assay for interferon regulatory factor 3 (IRF3), and surface plasmon resonance (SPR) indicated that some compounds had multiple interactions with STING at different concentrations. The assay disclosed herein enables determination of half maximum effective concentration (EC₅₀) for compounds showing one or more interactions, which aids in discovery of compounds capable of acting as STING antagonist and/or agonist drugs.

### Example 2. Microscale Thermophoresis (MST) Analysis of STING Modulators

### General Procedure 1

Nanotemper Monolith NT.115 labeled thermophoresis machine (NanoTemper Technologies GmbH, München, Germany) was used with standard treated capillary tubes using samples comprised of labeled protein and titrations of small molecule in 1×PBS. Microscale thermophoresis (MST) experiments were conducted in triplicate mixing 200 nM protein with 100 nM dye and allowing to sit at room temperature for 30 minutes followed by centrifugation on Ni-NTA 488 labeled His-labeled STING. Detection of the protein was performed using the blue detection channel with LED excitation power set to 90% and MST set to high allowing 3 s prior to MST on to check for initial fluorescence differences, 25 s for thermophoresis, and 3 s for regeneration after MST off. Analysis was performed using M.O. Affinity Analysis Software with difference between initial fluorescence measured in the first 5 s as compared with thermophoresis at 15 s at 15 different analyte concentrations ranging from 15 nM to 1 mM and exported into Graphpad Prism v.8 using a Log inhibitor v. response 4 parameter fit.

### General Procedure 2

Nanotemper Monolith NT.115 labeled thermophoresis machine (NanoTemper Technologies GmbH, München, Germany) was used with standard treated capillary tubes using samples comprised of labeled protein and titrations of small molecule in 1×HBS-P. Microscale thermophoresis (MST) experiments were conducted in triplicate mixing 200 nM protein with 100 nM dye and allowing to sit at room temperature for 1 hour followed by centrifugation on Ni-NTA 488 labeled His-labeled STING. Detection of the protein was performed using the blue detection channel with LED excitation power set to 90% and MST set to high allowing 3 s prior to MST on to check for initial fluorescence differences, 25 s for thermophoresis, and 3 s for regeneration after MST off. Analysis was performed using M.O. Affinity Analysis Software with difference between initial fluorescence measured in the first 5 s as compared with thermophoresis at 15 s at 15 different analyte concentrations ranging from 100 zM to 100 M and exported into Graphpad Prism v.8 using a Log inhibitor v. response 4 parameter fit.

### Results

Microscale Thermophoresis (MST) was performed on compounds incubating with 50 nM 2,3-cGAMP. Confidence is categorized as follows: 1=low confidence (<2 MST shift unit-non-statistical); 2=mid confidence (2-3 MST shift units-intermediate); 3=high confidence (>2 MST shift units-statistical). Dissociation constants are categorized as follows: A<1 pM; 1 pM≤B<100 pM; 100 pM≤C<10 μM; 10 μM≤D<100 μM. The results are shown in Table A.

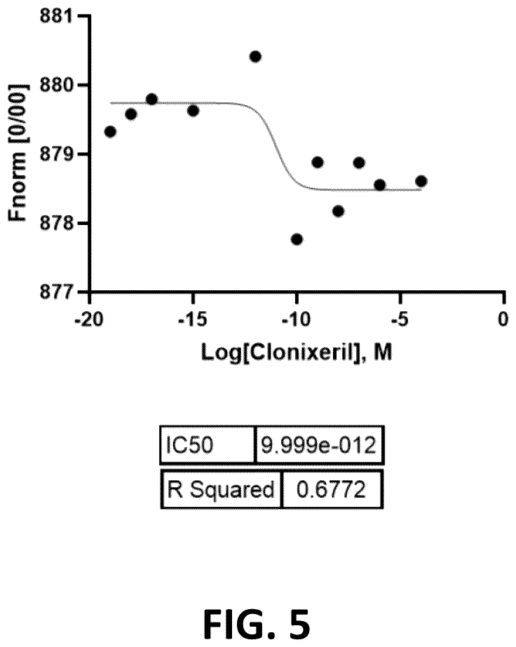

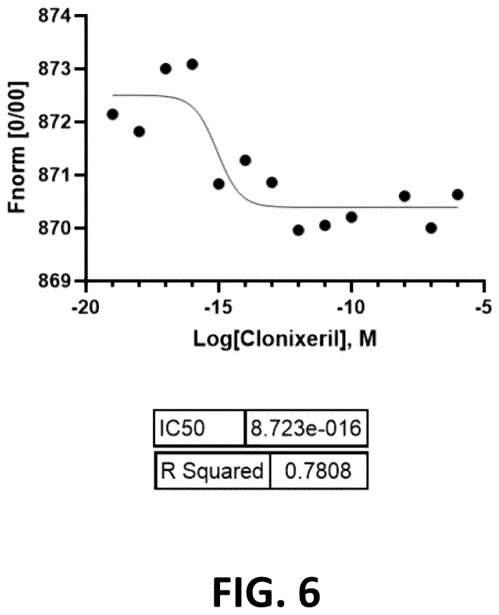

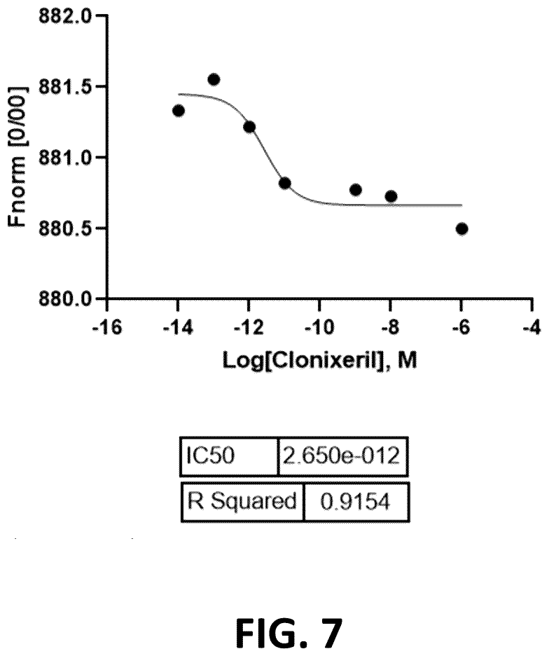

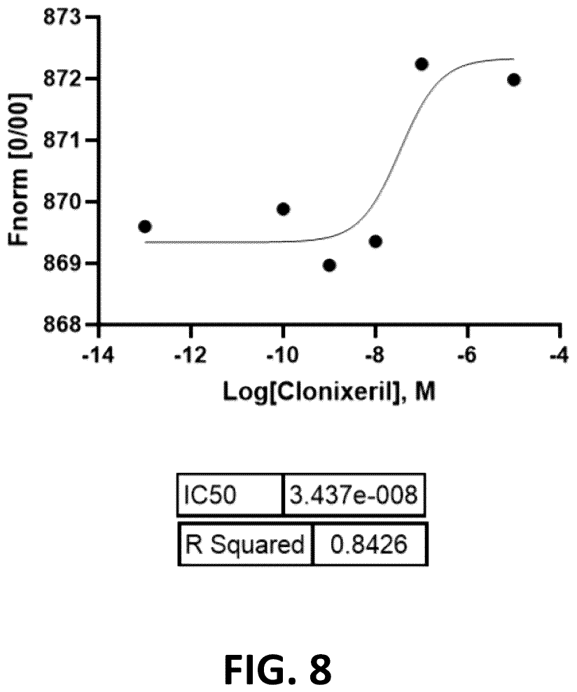

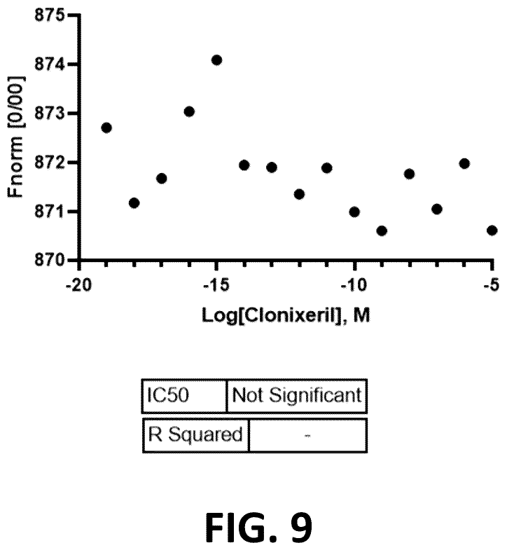

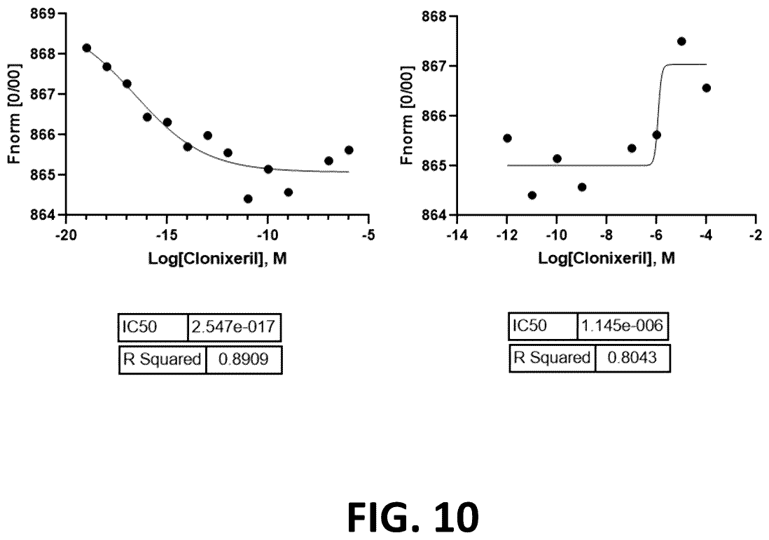

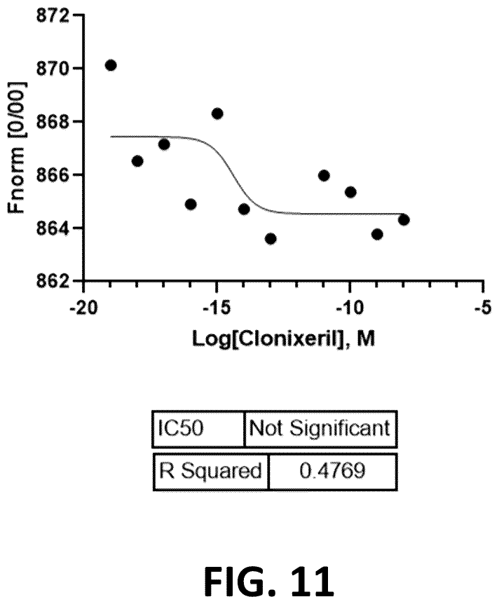

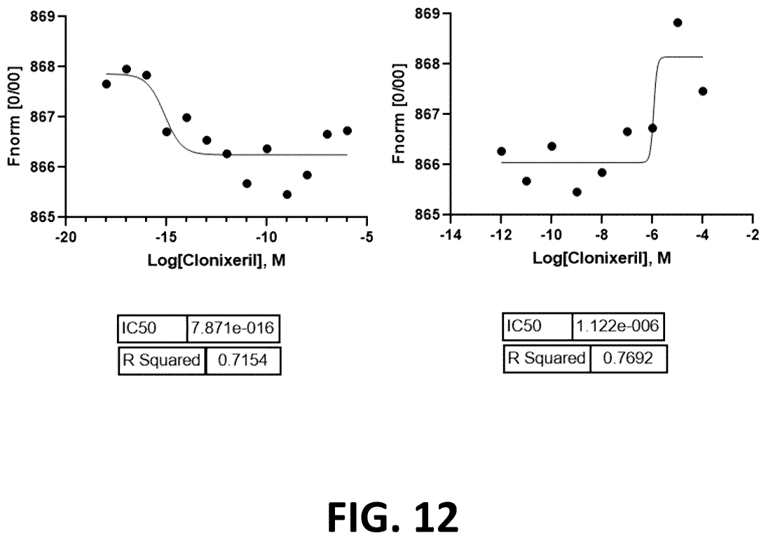

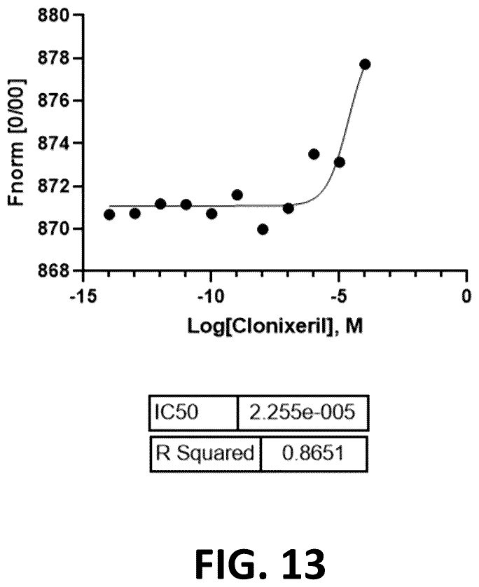

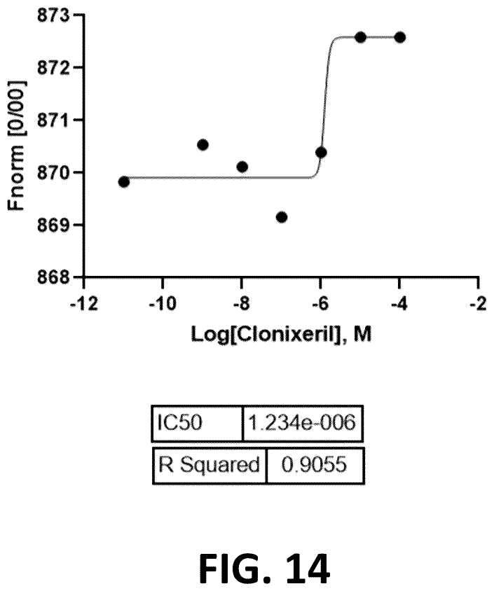

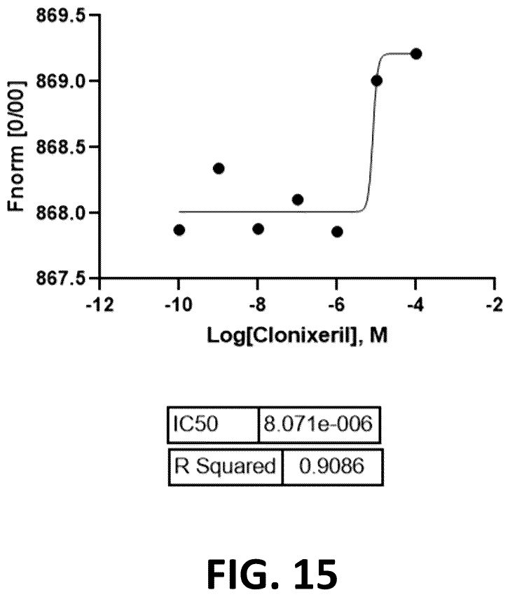

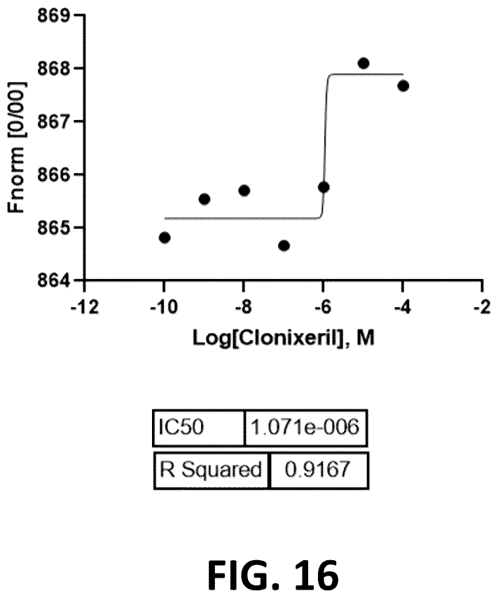

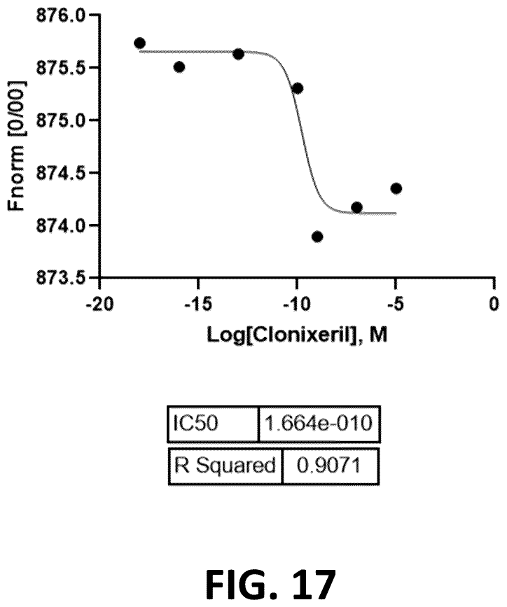

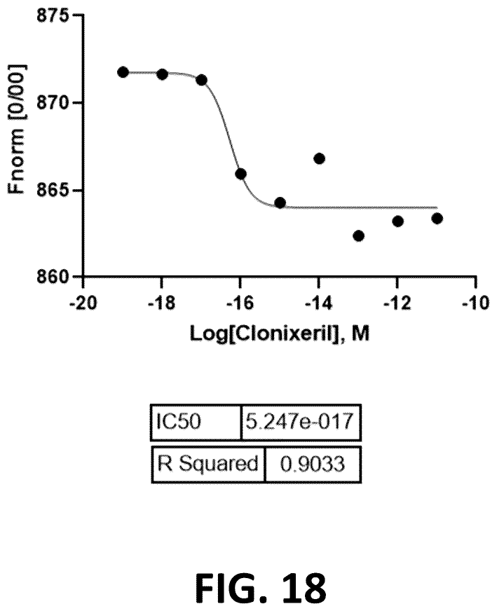

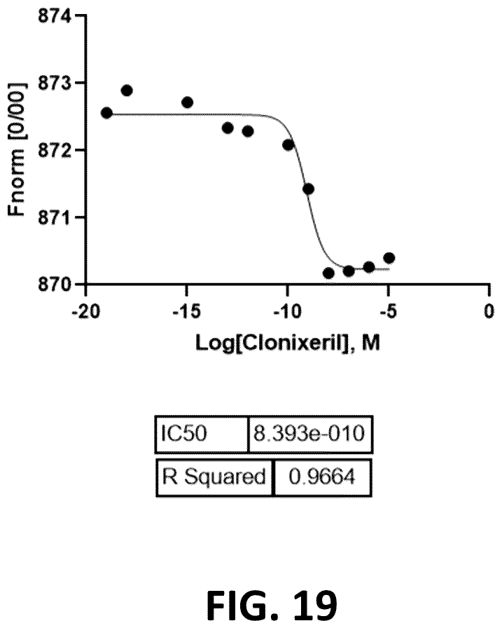

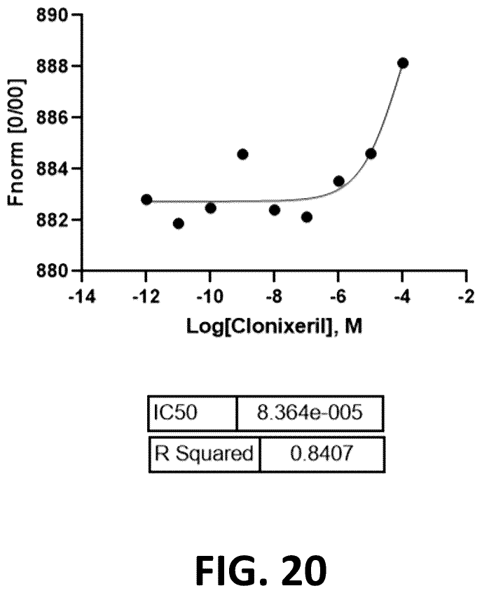

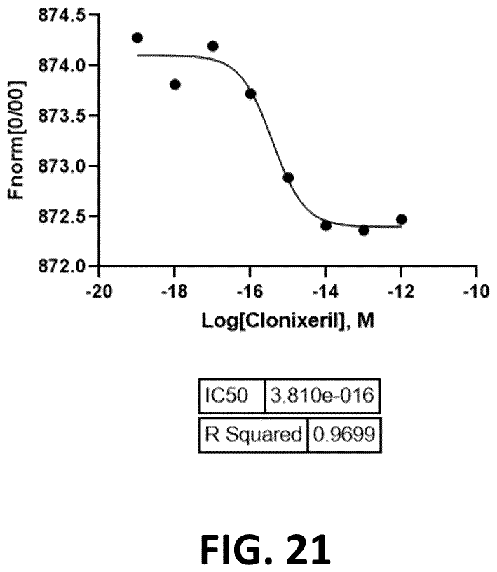

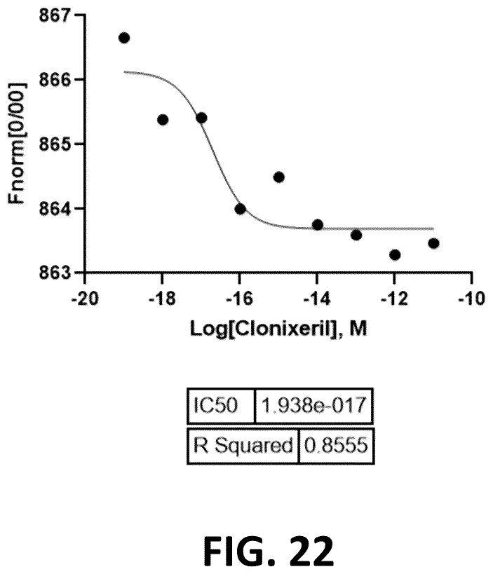

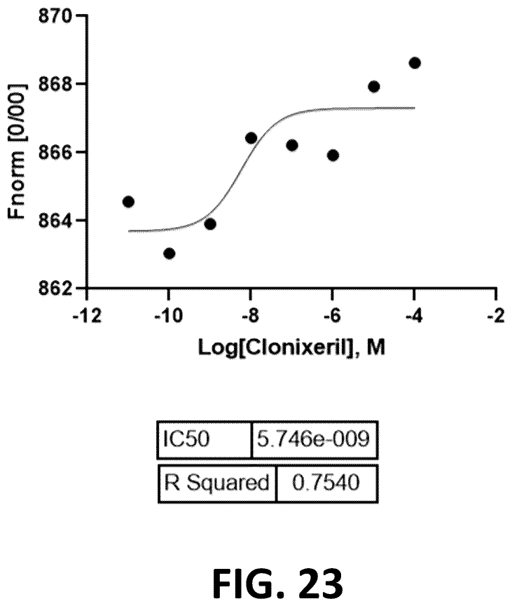

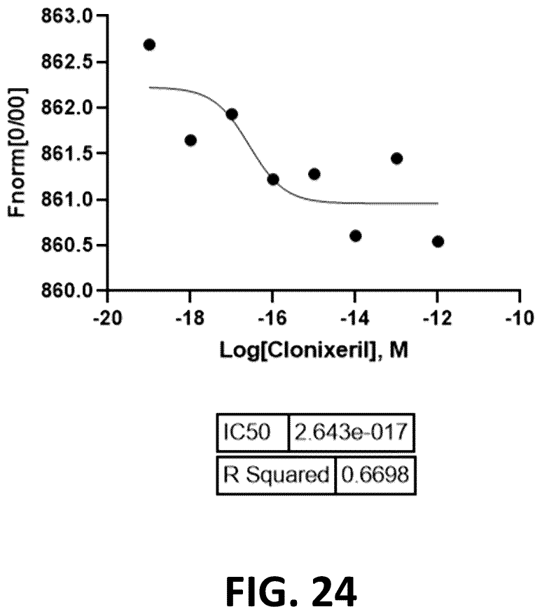

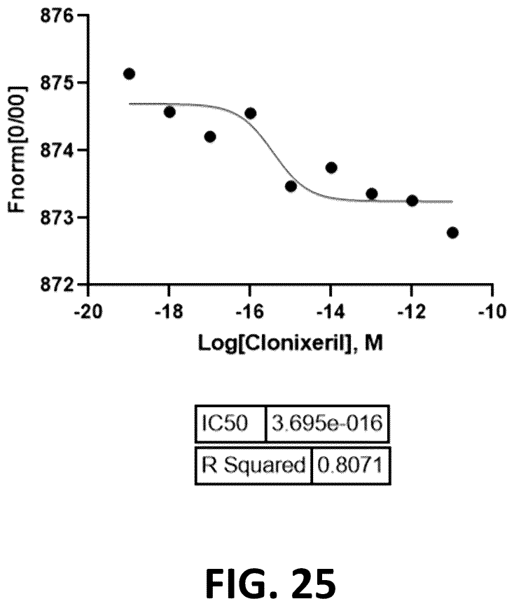

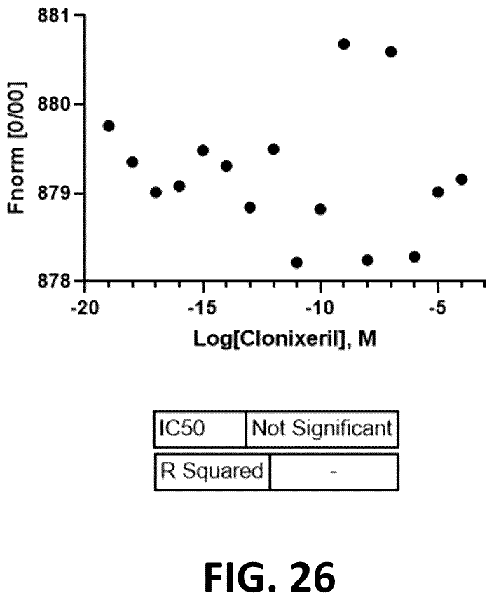

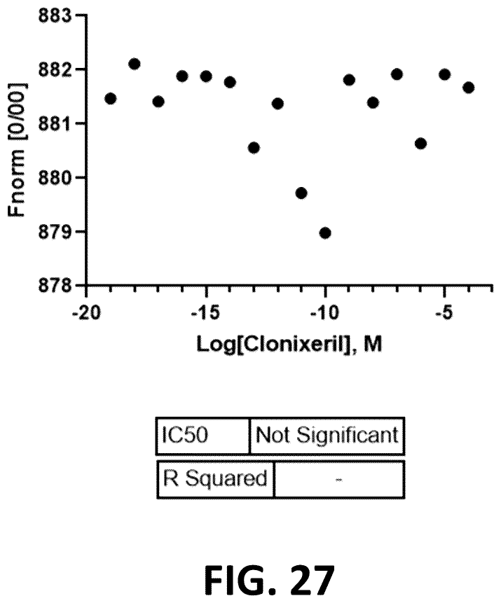

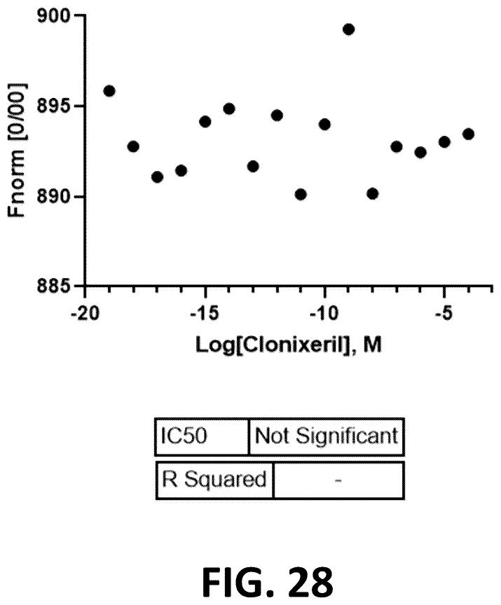

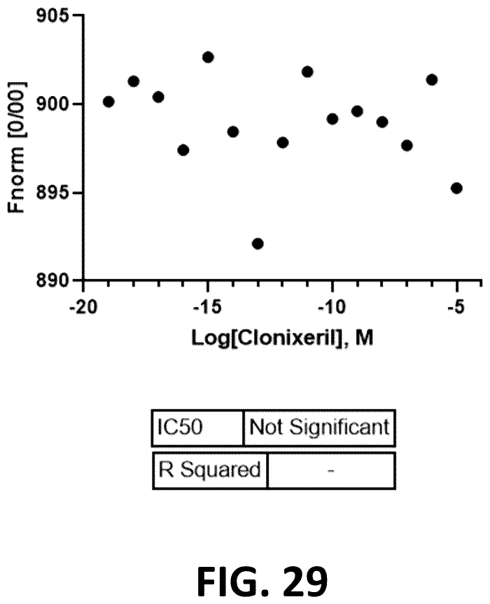

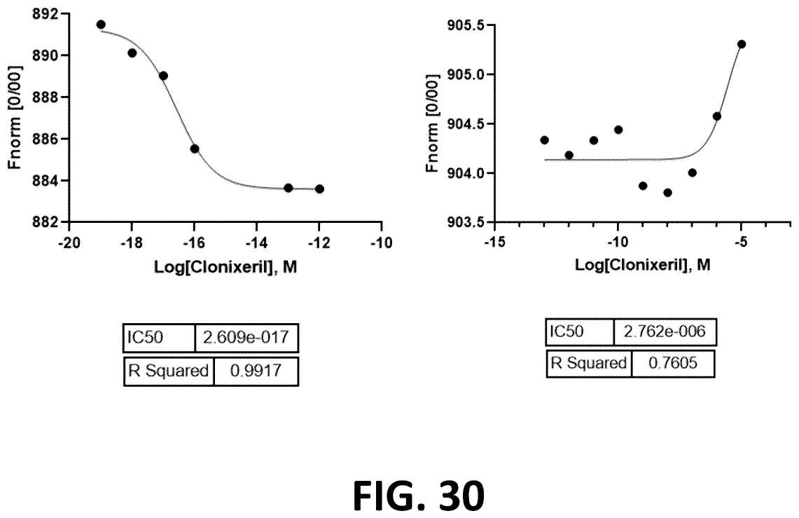

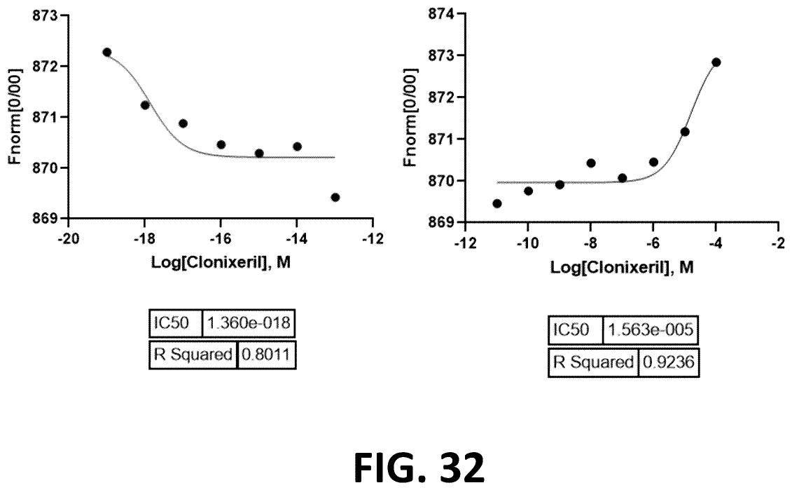

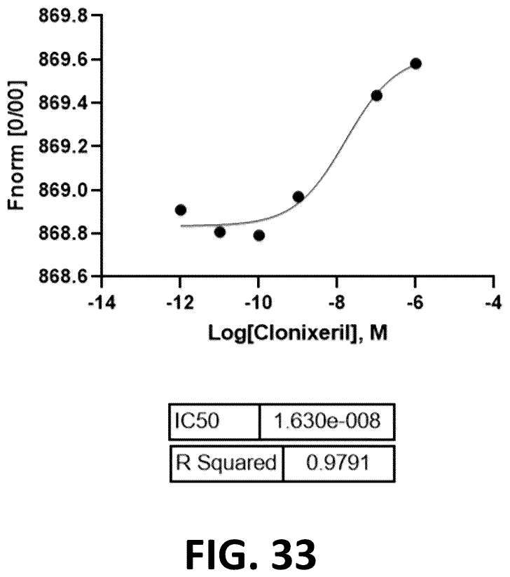

TABLE A
Dissociation Constants and Dose-Response Curves of Reference Compounds

| | | Dose Response | Dissociation | |
|---|---|---|---|---|
| Compound | Structure | Curve | Constant | Confidence |
| 1 | 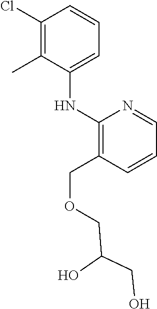 [SMILES: Cc1c(Cl)cccc1Nc1ncccc1COCC(O)CO] | FIG. 5 | B | 1 |
| 4 | 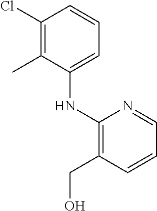 [SMILES: Cc1c(Cl)cccc1Nc1ncccc1CO] | FIG. 6 | A | 2 |
| 8 | 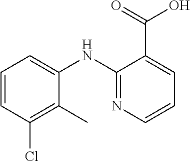 [SMILES: Cc1c(Cl)cccc1Nc1ncccc1C(=O)O] | FIG. 9 | No Binding | 1 |
| 9 | 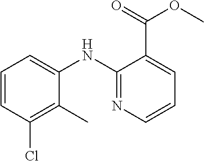 [SMILES: COC(=O)c1cccnc1Nc1cccc(Cl)c1C] | FIG. 10 | K_{d1}: C; K_{d2}: A | 3 |
| 10 | 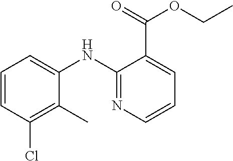 [SMILES: CCOC(=O)c1cccnc1Nc1cccc(Cl)c1C] | FIG. 11 | Low R2 | 1 |
| 11 | 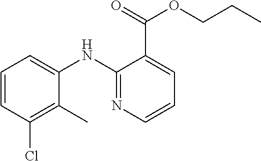 [SMILES: CCCOC(=O)c1cccnc1Nc1cccc(Cl)c1C] | FIG. 12 | K_{d1}: C; K_{d2}: A | 1 |
| 13 | 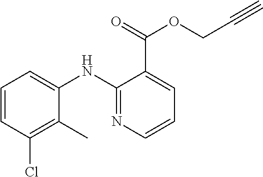 [SMILES: C#CCOC(=O)c1cccnc1Nc1cccc(Cl)c1C] | FIG. 13 | D | 2 |
| 14 | 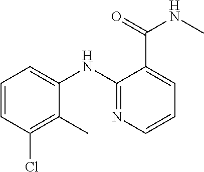 [SMILES: CNC(=O)c1cccnc1Nc1cccc(Cl)c1C] | FIG. 14 | C | 2 |
| 15 | 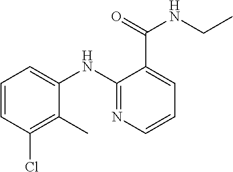 [SMILES: CCNC(=O)c1cccnc1Nc1cccc(Cl)c1C] | FIG. 15 | C | 1 |
| 16 | 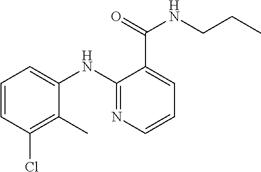 [SMILES: CCCNC(=O)c1cccnc1Nc1cccc(Cl)c1C] | FIG. 16 | C | 2 |
| 17 | 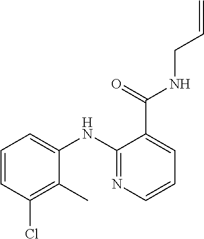 [SMILES: C=CCNC(=O)c1cccnc1Nc1cccc(Cl)c1C] | FIG. 17 | C | 1 |
| 19 | 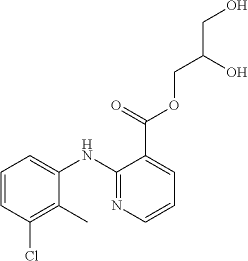 [SMILES: Cc1c(Cl)cccc1Nc1ncccc1C(=O)OCC(O)CO] | FIG. 18 | A | 1 |
| 21 | 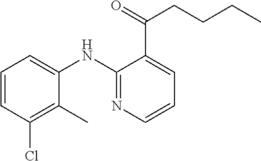 [SMILES: CCCCC(=O)c1cccnc1Nc1cccc(Cl)c1C] | FIG. 19 | C | 2 |
| 23 | 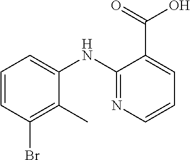 [SMILES: Cc1c(Br)cccc1Nc1ncccc1C(=O)O] | FIG. 20 | D | 3 |
| 24 | 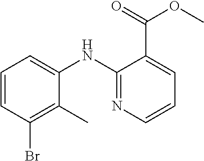 [SMILES: COC(=O)c1cccnc1Nc1cccc(Br)c1C] | FIG. 21 | B | 1 |
| 26 | 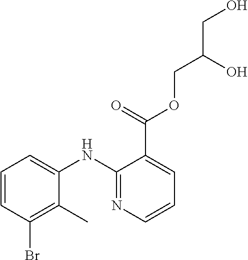 [SMILES: Cc1c(Br)cccc1Nc1ncccc1C(=O)OCC(O)CO] | FIG. 22 | A | Not available |
| 27 | 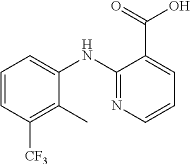 [SMILES: Cc1c(Nc2ncccc2C(=O)O)cccc1C(F)(F)F] | FIG. 23 | C | 3 |
| 29 | 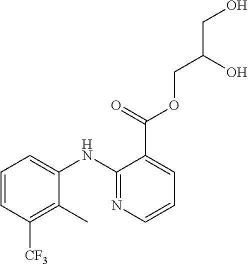 [SMILES: Cc1c(Nc2ncccc2C(=O)OCC(O)CO)cccc1C(F)(F)F] | FIG. 24 | A | Not available |
| 30 | 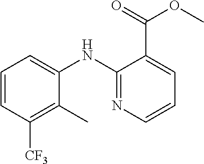 [SMILES: COC(=O)c1cccnc1Nc1cccc(C(F)(F)F)c1C] | FIG. 25 | B | Not available |
| 32 | 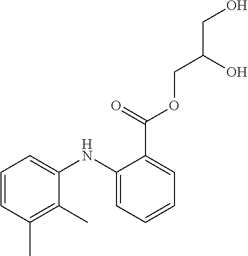 [SMILES: Cc1cccc(Nc2ccccc2C(=O)OCC(O)CO)c1C] | FIG. 26 | Not available | Not available |
| 33 | 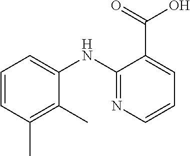 [SMILES: Cc1cccc(Nc2ncccc2C(=O)O)c1C] | FIG. 27 | Not available | Not available |
| 35 | 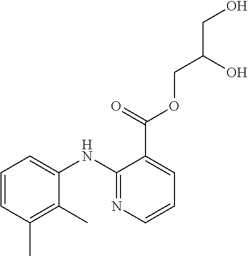 [SMILES: Cc1cccc(Nc2ncccc2C(=O)OCC(O)CO)c1C] | FIG. 28 | Not available | Not available |
| 36 | 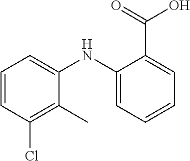 [SMILES: Cc1c(Cl)cccc1Nc1ccccc1C(=O)O] | FIG. 29 | No Binding | 1 |
| 38 | 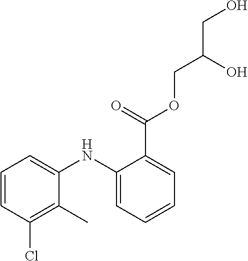 [SMILES: Cc1c(Cl)cccc1Nc1ccccc1C(=O)OCC(O)CO] | FIG. 30 | K_{d1}: C; K_{d2}: A | 1 |
| 39 | 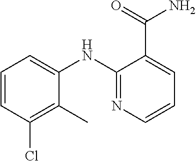 [SMILES: Cc1c(Cl)cccc1Nc1ncccc1C(N)=O] | FIG. 27 | Not available | Not available |
| 40 | 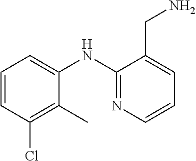 [SMILES: Cc1c(Cl)cccc1Nc1ncccc1CN] | FIG. 32 | K_{d1}: C; K_{d2}: A | Not available |
| 41 | 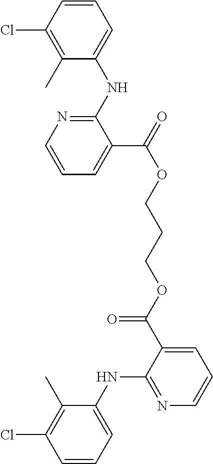 [SMILES: Cc1c(Cl)cccc1Nc1ncccc1C(=O)OCCCOC(=O)c1cccnc1Nc1cccc(Cl)c1C] | FIG. 33 | C | 1 |
| 42 | 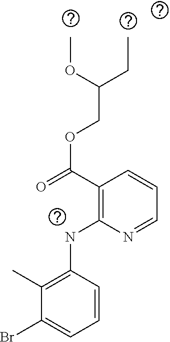 [SMILES: CCC(COC(=O)c1cccnc1Nc1cccc(Br)c1C)OC] | Not available | A | 2 |
| 43 | 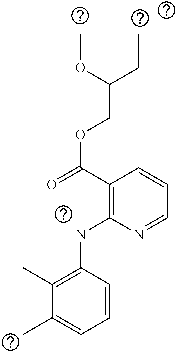 [SMILES: CCC(COC(=O)c1cccnc1Nc1cccc(C)c1C)OC] | Not available | A | 1 |
| 44 | 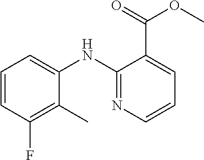 [SMILES: COC(=O)c1cccnc1Nc1cccc(F)c1C] | Not available | B | 1 |
| 45 | 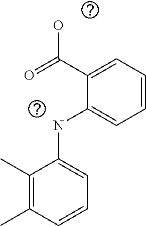 [SMILES: Cc1cccc(Nc2ccccc2C(=O)O)c1C] | Not available | No Binding | 1 |
| 46 | 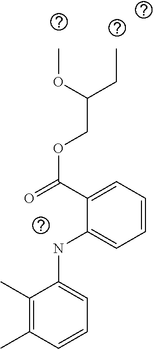 [SMILES: CCC(COC(=O)c1ccccc1Nc1cccc(C)c1C)OC] | Not available | No Binding | 1 |
| 47 | 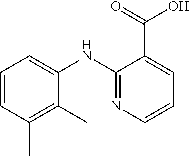 [SMILES: Cc1cccc(Nc2ncccc2C(=O)O)c1C] | Not available | No Binding | 1 |
| 48 | 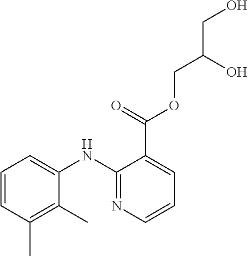 [SMILES: Cc1cccc(Nc2ncccc2C(=O)OCC(O)CO)c1C] | Not available | No Binding | 1 |
| 49 | 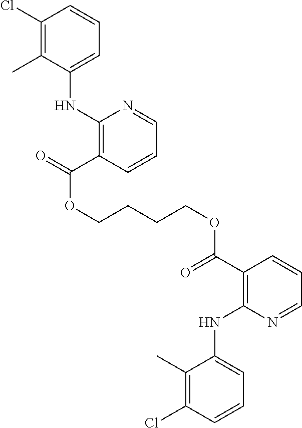 [SMILES: Cc1c(Cl)cccc1Nc1ncccc1C(=O)OCCCCOC(=O)c1cccnc1Nc1cccc(Cl)c1C] | FIG. 7 | B | 1 |
| 50 | 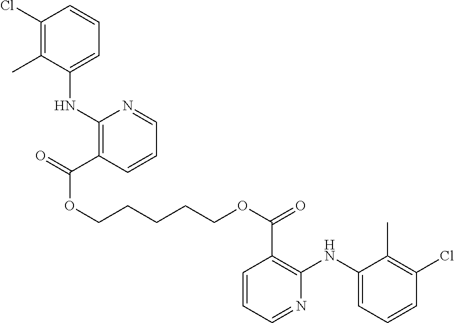 [SMILES: Cc1c(Cl)cccc1Nc1ncccc1C(=O)OCCCCCOC(=O)c1cccnc1Nc1cccc(Cl)c1C] | FIG. 8 | C | 3 |
| 52 | 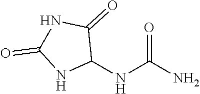 [SMILES: NC(=O)NC1NC(=O)NC1=O] | Not available | K_{d1}: C; K_{d2}: A | 3 |

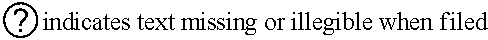

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A method for detecting the affinity between stimulator of interferon genes (STING) and a compound, the method comprising:
(a) contacting STING with 2′,3′-cGAMP to generate a first mixture, wherein the mixture comprises a detectable label;
(b) contacting the first mixture with the compound to form a second mixture; and
(c) detecting the detectable label using a microscale thermophoresis (MST) instrument.

2. The method of claim 1, wherein the first mixture is a buffered solution.

3. The method of claim 1, wherein the first mixture is a solution in 4-(2-hydroxyethyl)-1-piperazineethanesulfonic acid (HEPES) buffered saline surfactant P20 (HBS-P) buffer.

4. The method of claim 3, wherein the concentration of the STING in the HBS-P buffer is about 100 nM to about 300 nM.

5. The method of claim 3, wherein the concentration of the STING in the HBS-P buffer is about 200 nM.

6. The method of claim 1, wherein step (a) comprises contacting a dye with STING.

7. The method of claim 6, wherein the dye is a fluorescent dye.

8. The method of claim 7, wherein the fluorescent dye is NTAAtto 488 dye.

9. The method of claim 1, wherein the detectable label is a fluorescent label.

10. The method of claim 1, wherein contacting the first mixture with the compound comprises contacting the first mixture with a buffered solution of the compound.

11. The method of claim 1, wherein contacting the first mixture with the compound comprises contacting the first mixture with a solution of the compound in 4-(2-hydroxyethyl)-1-piperazineethanesulfonic acid (HEPES) buffered saline surfactant P20 (HBS-P) buffer.

12. The method of claim 10, wherein the solution comprises about 1% dimethylsulfoxide (DMSO).

13. The method of claim 10, wherein the concentration of the buffered solution of the compound is less than 200 μM.

14. The method of claim 10, wherein the concentration of the buffered solution of the compound is less than 1 μM.

15. The method of claim 10, wherein the concentration of the buffered solution of the compound is less than 1 nM.

16. The method of claim 10, wherein the concentration of the buffered solution of the compound is less than 1 μM.

17. The method of claim 10, wherein the concentration of the buffered solution of the compound is less than 1 fM.

18. The method of claim 10, wherein the concentration of the buffered solution of the compound is less than 1 aM.

19. The method of claim 10, wherein the concentration of the buffered solution of the compound is less than 1 zM.

20. The method of claim 1, wherein detecting the detectable label comprises measuring the normalized fluorescence as a function of the concentration of the solution of the compound.

21. The method of claim 1, wherein the compound is a compound of Formula (I), (II), (III), or (IV).

22. The method of claim 1, wherein the compound is a compound selected from the compounds disclosed in Table A.
